(19) United States
(12) Patent Application Publication
(10) Pub. No.: US 2025/0327804 A1
(43) Pub. Date: Oct. 23, 2025

(54) Methods and Compositions for Oral Cancer Risk

(71) Applicant: Vigilant Biosciences, Inc., Coral Springs, FL (US)

(72) Inventors: Dragan Nebrigic, Austin, TX (US); Zahra Mardy, Austin, TX (US); Geoffrey Landis, Carlsbad, CA (US)

(73) Assignee: Vigilant Biosciences, Inc., Coral Springs, FL (US)

(21) Appl. No.: 19/122,111

(22) PCT Filed: Oct. 17, 2023
(86) PCT No.: PCT/US2023/035308

§ 371 Date: Apr. 17, 2025

Related U.S. Application Data
(60) Provisional application No. 63/416,804, filed on Oct. 17, 2022.
(60) Provisional application No. 63/466,802, filed on May 16, 2023.

(51) Int. Cl. G01N 33/574

(52) U.S. Cl. CPC G01N 33/57407; G01N 2333/4704; G01N 2333/71

(57) ABSTRACT

This disclosure relates to methods and compositions for quantitative risk assessment of lesions in the oral cavity. The risk assessment tools can be used at point of care facilities. This disclosure provides methods and compositions for effective risk assessment of life-threatening or irreversibly debilitating human disease or conditions including oral cancer.

### CROSS-REFERENCE TO RELATED APPLICATIONS

This application claims the benefit of U.S. provisional patent application No. 63/416,804 filed on 17 Oct. 2022 entitled “SYSTEM AND METHOD FOR SUSPICIOUS ORAL LESIONS RISK ASSESSMENT” (attorney docket 015225-005PV1) and U.S. provisional patent application No. 63/466,802 filed on 16 May 2023 entitled “METHODS AND COMPOSITIONS FOR ORAL CANCER RISK” (attorney docket 015225-008PV1), both applications of which are hereby incorporated by reference in their entirety as if fully set forth herein.

### TECHNICAL FIELD

This invention relates to methods and compositions for quantitative, pre-diagnostic, point of care oral cancer risk assessment. Oral cancer risk is assessed using biomarkers p16 and EGFR in the whole saliva. A point of care device determines biomarker levels and determines a risk assessment along with clinical factors.

### BACKGROUND

Oral potentially malignant disease (OPMD) is a heterogeneous group of oral mucosal lesions associated with an increased risk of malignant transformation to cancer. These conditions can progress to oral cancer and life-threatening or irreversibly debilitating disease.

At present, a visual oral examination (VOE) is the conventional method used to identify oral mucosal abnormalities and/or lesions.

Drawbacks of visual oral examination include a high level of dependence on the experience of the health care provider. For example, several OPMD conditions such as white or red lesions and persistent ulcers are often indistinguishable by VOE at their clinical presentation.

Moreover, drawbacks of current adjunctive tools such as toluidine staining and light-based autofluorescence are lack of the necessary accuracy to detect disease.

What is needed are methods for detection and early detection or assessment of a risk of oral potentially malignant disease.

There is an urgent need for methods for health care providers to assess the risk of OPMD, or to diagnose, prognose, and/or monitor oral abnormalities and lesions. Methods are needed to improve detection, treatment and survival of oral cancer. There is a need for early diagnosis of oral cancer risk to improve treatment and minimize negative impacts on quality of life from invasive surgical intervention.

### BRIEF SUMMARY

This invention provides methods and compositions for quantitative risk assessment of lesions in the oral cavity. The risk assessment tools can be used at point of care facilities.

This disclosure provides methods and compositions for effective risk assessment of life-threatening or irreversibly debilitating human disease or conditions including oral cancer.

The methods of this invention can be used with or without conventional oral visual examination.

Methods of this invention can use patient saliva and provide effective risk assessment of oral cancer.

In some embodiments, this invention may utilize a lateral flow-based, multiplexed biomarker sensor device. Multiple independent biomarkers may be used for testing purposes.

In certain embodiments, methods of this invention may utilize p16 and EGFR biomarkers.

Embodiments of this invention can provide multi-biomarker, non-invasive, inexpensive, and easy to perform point-of-care testing methods with improved sensitivity and specificity that represent a breakthrough in early detection of oral cancer risk.

Embodiments of this invention include the following:

A method for assessing a risk level of, diagnosing, prognosing, or treating an oral cancer in a subject, the method comprising detecting biomarker levels in a saliva sample from the subject; and assessing the risk level of the oral cancer using the biomarker levels in combination with clinical factors of the subject. The treating the oral cancer can be based on the assessed risk level, diagnosis, or prognosis. The biomarkers may comprise p16 and EGFR. The oral mucosal abnormality may be an oral lesion, an oral cancer, a dysplasia, an early-stage cancer, or a pre-malignant disorder. The oral cancer may be located in any of a lip tissue, a gum tissue, a jaw tissue, a tongue tissue, or an oropharyngeal tissue. The method above may be a point of care risk assessment test. The sample can be obtained and the method performed in a point of care setting. The method can be performed in a point of care setting with a test duration of 3-5 minutes, or 5-50 minutes, or about 20 minutes. The saliva sample may be prepared from filtered whole saliva in a buffer solution. The saliva sample can be stored for less than 24 hours in a refrigerator, or is frozen and thawed no more than once. The method may provide an early detection of oral cancer or a risk of oral cancer in a subject. The treating can include any one or more of surgery, therapeutic radiation, chemotherapy, and drug therapy. The method can reduce a need for invasive or debilitating cancer treatment.

The method above, wherein the assessing a risk level of, diagnosing, prognosing, or treating comprises a logic combination of a sensor fusion function and a risk factor function to provide an output.

The method above, wherein the method is performed after visualizing an oral mucosal abnormality in the subject by a trained healthcare provider.

The method above, wherein the clinical factors comprise age, gender, alcohol use, tobacco use, and presence of a non-oral diagnosed cancer.

The method above, wherein the wherein the assessing a risk level of, diagnosing, prognosing, or treating results in a greater sensitivity, specificity, or predictive value as compared to visual oral examination alone.

The method above, wherein the assessing a risk level of, diagnosing, prognosing, or treating provides a specificity of at least 70%, or at least 80%, or at least 85%.

The method above, wherein the assessing a risk level of, diagnosing, prognosing, or treating provides a sensitivity of at least 70%, or at least 80%, or at least 85%.

The method above, wherein the assessing a risk level of, diagnosing, prognosing, or treating provides a negative or positive predictive value of at least 70%, or at least 80%, or at least 85%, or at least 90%.

The method above, wherein p16 is detected in a biologically relevant range from 0 to 160 ng/mL, or in a range of C5th to C95th percentile from 18.37 to 64.20 ng/ml, respectively.

The method above, wherein EGFR is detected in a biologically relevant range from 0 to 25 ng/ml, or in a range of C5th to C95th percentile from 2.43 to 16.50 ng/ml, respectively.

The method above, wherein no significant interference is caused by bilirubin, a-amylase, IgA, lactoferrin, whole blood, pooled common bacteria, or pooled common viruses.

The method above, wherein the level of p16 is detected as being low when less than 10.5 ng/mL, as being moderate when greater than 10.5 ng/ml and less than 27.5 ng/mL, and as being elevated when greater than 27.5 ng/ml.

The method above, wherein the level of EGFR is detected as being low when less than 0.75 ng/mL, as being moderate when greater than 0.75 ng/ml and less than 1.4 ng/mL, and as being elevated when greater than 1.4 ng/ml. The sensor fusion function can be based on two lateral flow biomarker levels being a low, moderate, or elevated level for p16 and a low, moderate, or elevated level for EGFR. The risk factor function may be based on risk factors being subject age, gender, alcohol use, tobacco use, and presence of a non-oral diagnosed cancer, wherein the risk factor function is determined by logistic regression to provide three risk ranges being low, moderate, and elevated risk. The regression can take the highest value for a risk factor when the clinical data is not available. The output may be a treatment referral based on the logic combination of the sensor fusion function and the risk factor function, wherein the output is one of: no treatment referral due to low combined risk; clinician decision for treatment referral due to moderate combined risk; or treatment referral due to moderate or elevated combined risk. The subject can be immediately referred for cancer treatment based on moderate or elevated combined risk.

This invention further contemplates methods for diagnosing, prognosing or monitoring an oral cancer in a subject, the method comprising: measuring biomarker levels in a saliva sample from the subject; comparing the levels of the biomarkers to reference levels based on a control group of subjects; detecting differences in the biomarker levels between the subject and the control group; diagnosing, prognosing or monitoring the oral cancer in the subject based on the differences. The biomarkers may comprise p16 and EGFR. The oral mucosal abnormality may be an oral lesion, an oral cancer, a dysplasia, an early-stage cancer, or a pre-malignant disorder. The oral cancer can be located in any of a lip tissue, a gum tissue, a jaw tissue, a tongue tissue, or an oropharyngeal tissue. The method may be performed in a point of care setting with a test duration of 3-5 minutes, or 5-50 minutes, or about 20 minutes. The saliva sample can be prepared from filtered whole saliva in a buffer solution. The sample may be stored for less than 24 hours in a refrigerator, or is frozen and thawed no more than once. The method may provide an early detection of oral cancer or a risk of oral cancer in a subject. The method can reduce a need for invasive or debilitating cancer treatment. The control group may comprise subjects having an oral mucosal abnormality or oral cancer. The biomarker levels can be determined by steps comprising: detecting a test line and a control line on a lateral flow substrate; determining an area for each of the test line and the control line and eliminating non-line areas; and measuring the optical intensities of the test line and the control line.

The method above, wherein the diagnosing or prognosing may comprise a logic combination of a sensor fusion function and a risk factor function to provide an output.

The method above, wherein the method is performed after visualizing an oral mucosal abnormality in the subject by a trained healthcare provider.

The method above, wherein diagnosing or prognosing can be based on additional clinical factors comprising age, gender, alcohol use, tobacco use, and presence of a non-oral diagnosed cancer.

The method above, wherein the biomarker levels may be determined by steps comprising: detecting a test line and a control line on a lateral flow substrate; determining an area for each of the test line and the control line and eliminating non-line areas; and measuring the optical intensities of the test line and the control line.

The method above, wherein the diagnosing or prognosing can result in a greater sensitivity, specificity, or predictive value as compared to visual oral examination alone.

The method above, wherein the diagnosing or prognosing may provide a specificity of at least 70%, or at least 80%, or at least 85%.

The method above, wherein the diagnosing or prognosing may provide a sensitivity of at least 70%, or at least 80%, or at least 85%.

The method above, wherein the diagnosing or prognosing may provide a negative or positive predictive value of at least 70%, or at least 80%, or at least 85%, or at least 90%.

The method above, wherein p16 can be detected in a biologically relevant range from 0 to 160 ng/mL, or in a range of C5th to C95th percentile from 18.37 to 64.20 ng/mL, respectively.

The method above, wherein EGFR may be detected in a biologically relevant range from 0 to 25 ng/mL, or in a range of C5th to C95th percentile from 2.43 to 16.50 ng/ml, respectively.

The method above, wherein no significant interference may be caused by bilirubin, a-amylase, IgA, lactoferrin, whole blood, pooled common bacteria, or pooled common viruses.

The method above, wherein the level of p16 may be detected as being low when less than 10.5 ng/ml, as being moderate when greater than 10.5 ng/mL and less than 27.5 ng/mL, and as being elevated when greater than 27.5 ng/ml.

The method above, wherein the level of EGFR can be detected as being low when less than 0.75 ng/ml, as being moderate when greater than 0.75 ng/ml and less than 1.4 ng/mL, and as being elevated when greater than 1.4 ng/mL. The sensor fusion function may be based on two lateral flow biomarker levels being a low, moderate, or elevated level for p16 and a low, moderate, or elevated level for EGFR. The risk factor function can be based on risk factors being subject age, gender, alcohol use, tobacco use, and presence of a non-oral diagnosed cancer, wherein the risk factor function is determined by logistic regression to provide three risk ranges being low, moderate, and elevated risk. The regression may take the highest value for a risk factor when the clinical data is not available. The output can be a treatment referral based on the logic combination of the sensor fusion function and the risk factor function, wherein the output is one of: no treatment referral due to low combined risk; clinician decision for treatment referral due to moderate combined risk; or treatment referral due to moderate or elevated combined risk. The subject can be immediately referred for cancer treatment based on moderate or elevated combined risk.

This invention further provides methods for data acquisition, the method comprising: obtaining a saliva sample from a subject; filtering the saliva sample; adding the filtered saliva to a buffer solution; and measuring biomarker levels in the filtered saliva sample. The biomarkers may comprise proteomic p16 and EGFR. The biomarker levels can be measured on a lateral flow test strip using an optical reader. The filtering can remove particles having a range of size from 1 to 100 micrometers, or from 1 to 10 micrometers, or from 0.1 to 10 micrometers, or from 0.1 to 5 micrometers, or from 0.1 to 2 micrometers, or from 0.1 to 1 micrometers. The filtering can remove particles having a size greater than about 0.05 micrometers, or about 0.1 micrometers, or about 0.2 micrometers, or about 0.45 micrometers, or about 1 micrometers, or about 2 micrometers, or about 5 micrometers.

The method above, wherein the saliva sample is stored for less than 24 hours in a refrigerator, or is frozen and thawed no more than once.

The method above, wherein the buffer solution is filtered after adding the saliva.

The method above, wherein the method is performed in a point of care setting with a duration of 3-5 minutes, or 5-50 minutes, or about 20 minutes.

The method above, wherein biomarker p16 is detected in a biologically relevant range from 0 to 160 ng/ml, or in a range of C5th to C95th percentile from 18.37 to 64.20 ng/mL, respectively.

The method above, wherein biomarker EGFR is detected in a biologically relevant range from 0 to 25 ng/mL, or in a range of C5th to C95th percentile from 2.43 to 16.50 ng/ml, respectively.

The method above, wherein the measured biomarkers levels are accurate even in the presence of any one or more of bilirubin, a-amylase, IgA, lactoferrin, whole blood, pooled common bacteria, or pooled common viruses.

The method above, wherein the level of biomarker p16 is detected as being low when less than 10.5 ng/ml, as being moderate when greater than 10.5 ng/ml and less than 27.5 ng/ml, and as being elevated when greater than 27.5 ng/mL.

The method above, wherein the level of biomarker EGFR is detected as being low when less than 0.75 ng/ml, as being moderate when greater than 0.75 ng/ml and less than 1.4 ng/ml, and as being elevated when greater than 1.4 ng/ml.

The method above, wherein the subject presents an oral mucosal abnormality viewed by a health care provider.

The method above, wherein the oral mucosal abnormality is an oral lesion, an oral cancer, a dysplasia, an early-stage cancer, or a pre-malignant disorder.

The method above, wherein the oral mucosal abnormality is located in any of a lip tissue, a gum tissue, a jaw tissue, a tongue tissue, or an oropharyngeal tissue.

### BRIEF DESCRIPTION OF THE DRAWINGS

FIG. 1 shows a HPLC chromatograph of p16INK4a/CKD2a.

FIG. 2 shows a HPLC chromatograph of sEGFR.

FIG. 3 shows a ES-MS of p16INK4a/CKD2a.

FIG. 4 shows an SDS-PAGE analysis of rhEGFR detector and capture antibodies.

FIG. 5 shows an SDS-PAGE analysis of p16INK4a detector and capture antibodies.

FIG. 6 shows an SDS-PAGE gel of commercial p16INK4A.

FIG. 7 shows ELISA sandwich assays for binding characteristics (measured in OD450 nm) of EGFR and p16 antigens to matching antibodies. Upper curve shows results for EGFRAb-1 tethered to the surface of a microtiter plate well and EGFRAb-2 being free in solution to bind to the different epitopes of sEGFR antigens. The lower curve shows EGFRAb-2 being tethered the surface of the microtiter plate well and EGFRAb-1 being free in solution.

FIG. 8 shows an ELISA assay for p16 antigen when Abcam-2 is attached to the microtiter plate and Abcam-1 is used as the detector antibody.

FIG. 9 shows a sensorgram of the immobilization of the anti-EGFR mAb (Cetuximab) to the surface of the CM5 chip.

FIG. 10 shows a sensorgram of the sequential binding of rhEGFR to the CM5 chip-bound anti-EGFR mAb (Cetuximab), followed by the binding of the anti-EGFR mAb (EGFR. 1) to a second epitope on rhEGFR, forming the triplex of two antibodies bound to separate epitopes on the antigen (sandwich assay).

FIG. 11 shows a sensorgram of the immobilization of the p16mAb695 to the surface of the CM5 chip.

FIG. 12 shows a sensorgram of the sequential binding of p16INK4a to the CM5 chip-bound p16mAb695, followed by the binding of the p16mAb961 to a second epitope on p16INK4a, forming the triplex of two antibodies bound to separate epitopes on the antigen (sandwich assay).

FIG. 13 shows a dose-response curve of varying concentrations of p16 spiked into synthetic saliva.

FIG. 14 shows a dose-response curve of varying concentrations of EGFR spiked into synthetic saliva.

FIG. 15 shows a dose-response curve of p16 spiked into pooled whole human saliva.

FIG. 16 shows a dose-response curve of EGFR spiked into pooled whole human saliva.

FIG. 17 shows a dose-response curve of p16 spiked into pooled whole human saliva.

FIG. 18 shows a dose-response curve of EGFR spiked into pooled whole human saliva.

FIG. 19 shows an estimated moderate range of p16 measured when positive and negative pathology-confirmed results are overlapping.

FIG. 20 shows an estimated moderate range of EGFR measured when positive and negative pathology-confirmed results are overlapping.

FIG. 21 shows a logic table for combining a Clinical Risk Factor (CRF) assessment and a Sensor Fusion assessment.

FIG. 22 shows steps of a method for assessing a risk level of, or diagnosing, or prognosing an oral cancer.

FIG. 23 shows steps of a method for assessing a risk level of, or diagnosing, or prognosing an oral cancer.

FIG. 24 shows steps of a method for assessing a risk level of, or diagnosing, or prognosing an oral cancer with early detection.

FIG. 25 shows steps of a method for assessing a risk level of, or diagnosing, or prognosing, and/or treating an oral cancer.

FIG. 26 shows steps of a method for diagnosing, prognosing or monitoring an oral cancer.

FIG. 27 shows optical detection of p16 dose to optical units using a reader of this disclosure.

FIG. 28 shows optical detection of EGFR dose to optical units using a reader of this disclosure.

FIG. 29 shows optical detection of p16 dose to optical units using a reader of this disclosure.

FIG. 30 shows optical detection of EGFR dose to optical units using a reader of this disclosure.

### DETAILED DESCRIPTION OF THE INVENTION

This invention provides methods and compositions for performing a quantitative risk assessment of suspicious lesions in the oral cavity. The risk assessment tools can be operated by health care providers at point of care facilities including dentists, orthodontists, endodontists, periodontists, and oral medical providers such as oral surgeons and oral radiologists, as well as any hospital or radiology department.

This disclosure provides methods and compositions for effective risk assessment of life-threatening or irreversibly debilitating human disease or conditions including oral cancer.

In some embodiments, the methods and compositions of this invention can be combined with conventional oral visual examination in a health care provider's point of care facility or clinic.

A subject of this disclosure can be any human, or an adult human, or an adult human symptomatic of an oral abnormality or an oral cancer, or an adult human having an oral abnormality or an oral cancer who is asymptomatic.

In further embodiments, the methods and compositions of this invention can be used for symptomatic adult patients, who may present an oral mucosal abnormality visible to the health care provider.

In certain embodiments, methods and compositions of this invention can provide advantageously effective risk assessment of oral cancer as compared to conventional visual examination by a health care provider.

Methods of this disclosure can provide point-of-care measurements which are simplified and miniaturized. The methods disclosed herein can use patient saliva, which allows healthcare providers to perform a chairside examination. Methods of this invention can provide substantially improved pre-diagnostic and diagnostic capabilities.

In some embodiments, this invention may utilize a lateral flow-based, multiplexed biomarker sensor device. Methods of this invention can use multiple biomarkers for testing purposes. The biomarkers can be highly independent from each other for oral disease detection.

In certain embodiments, methods of this invention may utilize p16 and EGFR biomarkers.

In some aspects, methods of this this invention may utilize a reader integrated, quantitative risk assessment evaluation device. These methods can be applied for assessing a risk due to an oral abnormality or lesion and for detecting oral disease. Further, these methods can provide a rapid and timely integrated quantitative cancer risk assessment profile during a visit to a healthcare provider.

In additional aspects, this invention can result in a multiparametric biomarker cancer risk assessment signature, obtained rapidly after salivary content is dispensed onto a lateral flow device. Temporal effects of biomarker antibodies and salivary reaction may contain a multiparametric vector of information that can enable higher predictability of oral disease risk.

Embodiments of this invention can provide multi-biomarker, non-invasive, inexpensive, and easy to perform point-of-care testing methods with improved sensitivity and specificity that represent a breakthrough in early detection of oral cancer risk.

### Assessing Oral Cancer Risk

In some embodiments, oral cancer risk assessment can include a Clinical Risk Factor (CRF) assessment and a Sensor Fusion assessment.

A Clinical Risk Factor (CRF) assessment may include a multiparametric regression of several clinical risk factors.

In some embodiments, clinical risk factors include age, gender, alcohol use, tobacco use, and presence of other cancers.

A clinical risk factor assessment can be performed for oral cancer with whole saliva samples of positive and negative pathology-confirmed patients. A multiparametric regression can be used to determine coefficients for each clinical risk factor. Training and validation steps for the regression can be performed with the patient data.

In some embodiments, the clinical risk factor assessment can provide ranges for low, moderate, and elevated oral cancer risk.

A Sensor Fusion assessment may include quantitative lateral flow results based on biomarker measurements.

In some embodiments, a Sensor Fusion assessment may include measurements of P16 and EGFR biomarkers. A Sensor Fusion assessment may provide low, moderate, or elevated status for p16 biomarker. A Sensor Fusion assessment may provide low, moderate, or elevated status for EGFR biomarker.

Biomarkers used in methods of this invention can be proteomic biomarkers.

In further aspects, oral cancer risk assessment can include a combination of a Clinical Risk Factor (CRF) assessment and a Sensor Fusion assessment.

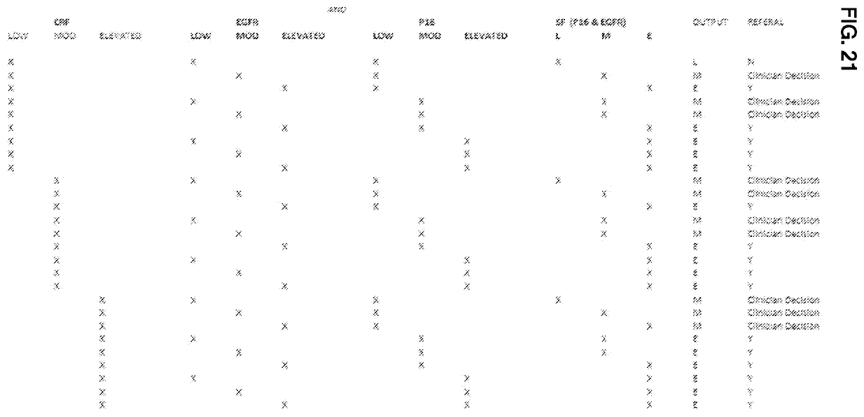

In certain embodiments, oral cancer risk assessment can include a combination of a Clinical Risk Factor (CRF) assessment and a Sensor Fusion assessment as shown in FIG. 21. As shown in FIG. 21, a logic table can be used to combine a Clinical Risk Factor (CRF) assessment and a Sensor Fusion assessment to provide a health care referral decision.

In further embodiments, oral cancer risk assessment can include a combination of a Clinical Risk Factor assessment and a Sensor Fusion assessment, where the clinical risk factors each provide a value and the Sensor Fusion biomarker levels each provide a value to be used in a multivariate statistical analysis based on Cox proportional hazards, machine learning, clustering analysis, classification and regression trees, random forest, principal component analysis, curds and whey analysis, discriminant analysis, stepwise regression, logistic regression, neural network analysis, vector machine analysis, and combinations thereof.

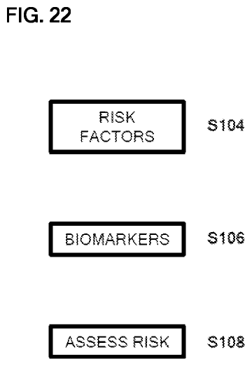

This invention contemplates methods as show in FIG. 22. In step S104, risk factors can be obtained and evaluated for entry into a non-transitory storage medium. In step S106, biomarker levels can be measured and entered into the non-transitory storage medium. In step S108, a risk of oral cancer can be determined from stored risk factors and biomarker levels.

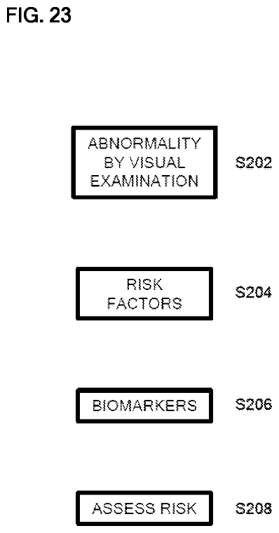

This invention further contemplates methods as show in FIG. 23. In step S202, an oral abnormality can be observed in a patient by a health care provider, solely by visual examination of the patient. In step S204, risk factors can be obtained and evaluated for entry into a non-transitory storage medium. In step S206, biomarker levels can be measured and entered into the non-transitory storage medium. In step S208, a risk of oral cancer can be determined from stored risk factors and biomarker levels.

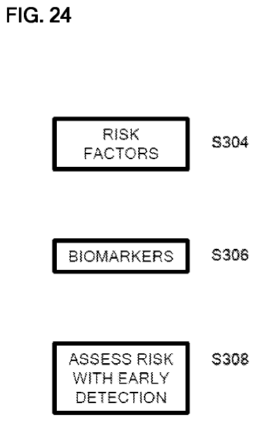

This invention also contemplates methods as show in FIG. 24. In step S304, risk factors can be obtained and evaluated for entry into a non-transitory storage medium. In step S306, biomarker levels can be measured and entered into the non-transitory storage medium. In step S308, a risk of oral cancer can be determined from stored risk factors and biomarker levels, where the risk assessment provides early detection of an oral cancer, either alone, or in combination with additional clinical work.

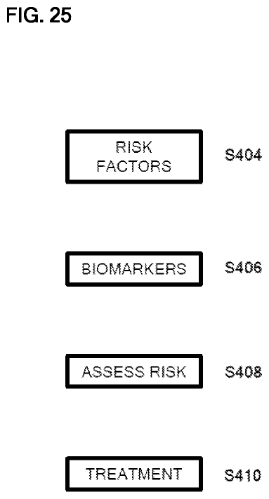

In addition, this invention contemplates methods as show in FIG. 25. In step S404, risk factors can be obtained and evaluated for entry into a non-transitory storage medium. In step S406, patient biomarker levels can be measured and entered into the non-transitory storage medium. In step S408, a risk of oral cancer in the patient can be determined from stored risk factors and biomarker levels. In step S410, an oral cancer or abnormality in the patient can be treated. Treatment can include any one or more of surgery, therapeutic radiation, chemotherapy, and drug therapy.

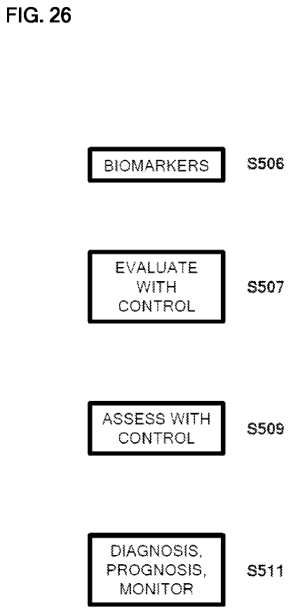

In further embodiments, this invention contemplates methods as show in FIG. 26. In step S506, biomarker levels can be measured and entered into a non-transitory storage medium. In step S507, stored biomarker levels can be compared for evaluation against a control group. In step S509, differences between the stored biomarker levels and levels of a control group can be detected. In step S511, a diagnosis, prognosis or monitoring of the oral cancer or abnormality in the subject can be performed based on the differences.

In certain embodiments, this invention further encompasses methods for data acquisition. The method for data acquisition can include patient biomarker levels. To obtain patient biomarker levels, a saliva sample may be taken from a patient. The saliva sample can be added to a buffer solution. The saliva sample can be filtered to remove particles of certain sizes from the saliva, before or after addition to the buffer solution. Patient biomarker levels can be measured in a filtered saliva sample, or a filtered saliva buffer solution.

In further aspects, the filtering of a saliva or buffer solution can remove particles having a range of size from 1 to 100 micrometers, or from 1 to 10 micrometers, or from 0.1 to 10 micrometers, or from 0.1 to 5 micrometers, or from 0.1 to 2 micrometers, or from 0.1 to 1 micrometers.

In additional aspects, the filtering of a saliva or buffer solution can remove particles having a size greater than about 0.05 micrometers, or about 0.1 micrometers, or about 0.2 micrometers, or about 0.45 micrometers, or about 1 micrometers, or about 2 micrometers, or about 5 micrometers.

### Data Collection

Embodiments of this invention include data collection via an optical reader system for lateral flow test strips (BEVIGILANT RAPID READER). The reader can detect p16 and EGFR biomarkers in the test cassette. The reader can detect low, medium, and high concentration ranges of the biomarkers.

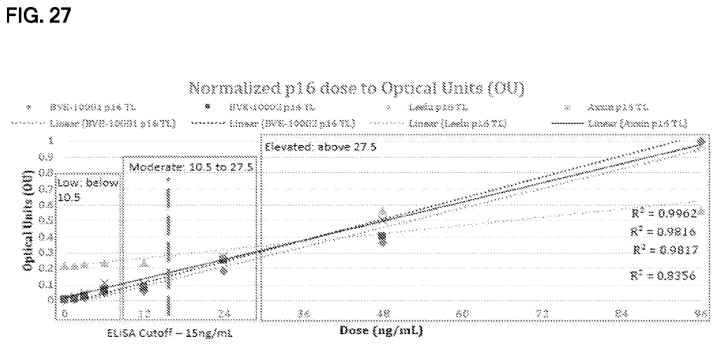

FIG. 27 shows optical detection of p16 dose to optical units using a reader of this disclosure.

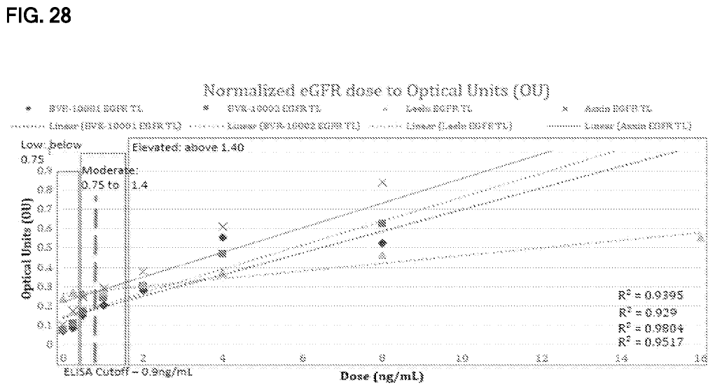

FIG. 28 shows optical detection of EGFR dose to optical units using a reader of this disclosure.

A reader of this disclosure can provide p16 test line R² of 0.981, and EGFR test line R² of 0.98. These values exceed that of known readers.

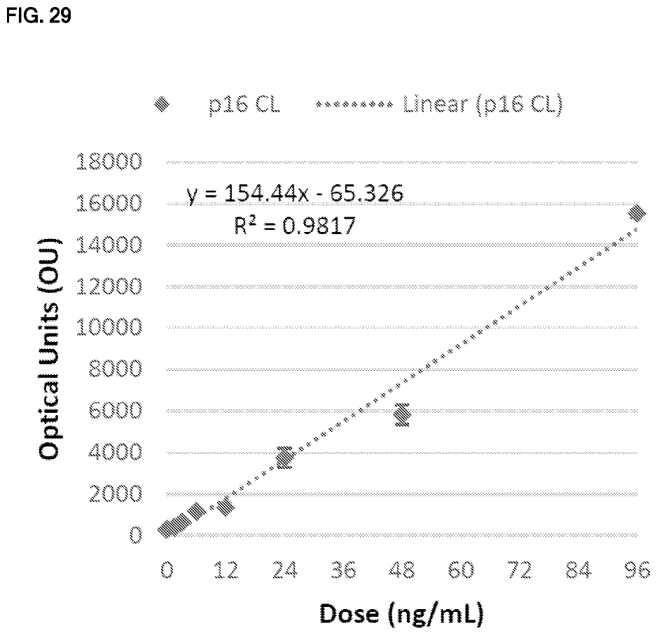

FIG. 29 shows optical detection of p16 dose to optical units using a reader of this disclosure.

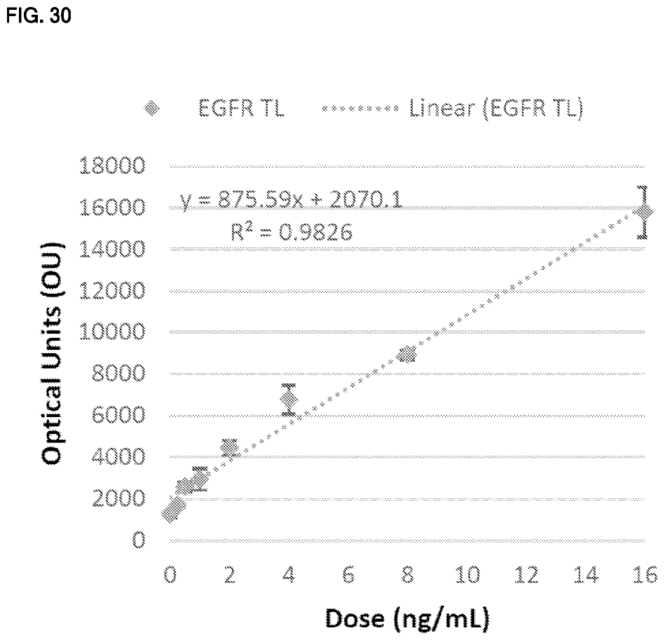

FIG. 30 shows optical detection of EGFR dose to optical units using a reader of this disclosure.

In some embodiments, this invention includes methods for data acquisition, in which a saliva sample can be obtained from a subject. The saliva sample may be filtered, before or after adding the saliva to a buffer solution. Biomarker levels can be measured in the filtered saliva sample. In certain embodiments, the biomarker levels may be measured on a lateral flow test strip using an optical reader.

In further embodiments, methods for data acquisition may include steps for
obtaining a saliva sample from a subject;
filtering the saliva sample;
adding the filtered saliva to a buffer solution; and
measuring biomarker levels in the filtered saliva sample.

The saliva sample may be stored for less than 24 hours in a refrigerator, or can be frozen and thawed, preferably no more than once.

Methods of this disclosure for data collection can be performed in a point of care setting. The duration of data collection, or a complete run of a test, may be from 3-5 minutes, or from 5-50 minutes, or about 20 minutes.

In certain embodiments, biomarker p16 can be detected in a biologically relevant range from 0 to 160 ng/ml, or in a range of C5th to C95th percentile from 18.37 to 64.20 ng/mL, respectively.

In further embodiments, biomarker EGFR can be detected in a biologically relevant range from 0 to 25 ng/ml, or in a range of C5th to C95th percentile from 2.43 to 16.50 ng/ml, respectively.

Methods of data collection of this disclosure can provide accurate biomarkers levels even in the presence of any one or more of bilirubin, a-amylase, IgA, lactoferrin, whole blood, pooled common bacteria, or pooled common viruses.

In some aspects, biomarker p16 may be detected as being low when less than 10.5 ng/ml, as being moderate when greater than 10.5 ng/ml and less than 27.5 ng/ml, and as being elevated when greater than 27.5 ng/ml.

In further aspects, biomarker EGFR may be detected as being low when less than 0.75 ng/ml, as being moderate when greater than 0.75 ng/ml and less than 1.4 ng/mL, and as being elevated when greater than 1.4 ng/mL.

Embodiments of this invention may utilize a subject who presents an oral mucosal abnormality viewed by a health care provider. The oral mucosal abnormality may be an oral lesion, an oral cancer, a dysplasia, an early-stage cancer, or a pre-malignant disorder. In some embodiments, the oral mucosal abnormality may be located in any of a lip tissue, a gum tissue, a jaw tissue, a tongue tissue, or an oropharyngeal tissue.

### Ligand Binding Assays

In some embodiments, validated ligand-binding assays for lateral flow reagents are used to ensure robust and consistent assay performance throughout. Ligand-binding assays can be enhanced by physico-chemical and biophysical characterization of reagents linked to assay performance.

Analytical techniques such as high-performance liquid chromatography electrospray mass spectroscopy (HPLC-ESMS), Surface Plasmon Resonance (SPR), Enzyme Linked Immunosorbent Assay (ELISA), and Sodium Dodecyl Sulfate-polyacrylamide Gel Electrophoresis (SDS-PAGE) assays can be used to verify and validate assay reagents.

In some embodiments, this invention provides reliable and precise batch-to-batch variability of reagents for use in a lateral flow device. In such embodiments, analytical results for antigens and antibodies were in accordance with highest standards for lateral flow systems.

### Devices

An embodiment of a device that may be used in a method of this invention may be a lateral flow device with a collection device. Examples of a collection device include SALETTO ORAL FLUID COLLECTION DEVICE (Porex Life Sciences Institute). The collection device may collect, filter, and deliver a saliva sample into a lateral flow device.

Examples of a lateral flow device can be based on utilization of colorimetric biomarker antibody pairs for each of P16, EGFR and a control line. A lateral flow device can include a lateral flow immunoassay cassette and a reader device.

A device for use in methods of this invention can provide a point of care quantitative oral cancer risk assessment. The device can provide pre-diagnostic or diagnostic information.

The methods of this invention can be utilized for symptomatic or asymptomatic adult patients. In some embodiments, methods of this invention can be utilized for symptomatic adult patients having an oral mucosal abnormality. The oral mucosal abnormality may be visible to a health-care provider by visual examination.

In operation, device for use in methods of this invention can receive patient data and clinical risk factors. A sample may be collected from a patient using a collection device. The sample may be filtered and combined with a buffer. The sample can be applied to a well on a test cassette. The test cassette can be placed in a positioning tray of a reader device. The device operation may be initiated to analyze the sample and provide a test result.

All publications including patents, patent application publications, and non-patent publications referred to in this description are each expressly incorporated herein by reference in their entirety for all purposes.

Although the foregoing disclosure has been described in detail by way of example for purposes of clarity of understanding, it will be apparent to the artisan that certain changes and modifications are comprehended by this disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

It is emphasized herein according to common practice the features of the drawings have arbitrary scale and are intended to cover similar features that may be arbitrarily expanded or reduced.

### EXAMPLES

### Example 1: Assessment of Oral Cancer Risk by the Methods of this Invention was Significantly Superior to Conventional Oral Visual Examination

Health care providers use oral visual examination to detect and characterize suspicious oral lesions. The assessment of oral cancer risk by this method does not provide high accuracy.

A blinded study was performed in multiple dentists' offices to evaluate abilities of dentists in determining patient risk by oral visual examination alone of suspicious lesions.

The cumulative results of the four participating dental offices, based solely on visual examination, are shown in Table 1.

TABLE 1
Cumulative results in dental offices
based solely on visual examination

| | CONDITION PRESENT | CONDITION ABSENT |
|---|---|---|
| POSITIVE | 143 | 71 |
| NEGATIVE | 63 | 127 |

The study showed that confidence in risk assessment by visual examination alone is relatively poor. As shown in Table 2, sensitivity of only up to 69% was achieved by visual examination alone.

TABLE 2
Assessment of oral cancer risk based solely on visual examination

| | VISUAL EXAM |
|---|---|
| SENSITIVITY | 69% |
| SPECIFICITY | 64% |
| NEGATIVE PREDICTIVE VALUE | 67% |
| POSITIVE PREDICTIVE VALUE | 67% |

Further, as shown in Table 2, specificity and predictive value did not exceed 67%.

By comparison, compositions and embodiments of this invention provided surprisingly improved sensitivity and specificity using ORAFUSION assay methods. As shown in Table 3, the methods of this invention provided sensitivity, specificity and predictive values surprisingly higher than for conventional assessment based solely on visual examination. As shown in Table 3, the methods of this invention achieved sensitivity of 91%.

TABLE 3
Assessment of oral cancer risk by health
care providers using ORAFUSION

| | ORAFUSION |
|---|---|
| SENSITIVITY | 91% |
| SPECIFICITY | 88% |
| NEGATIVE PREDICTIVE VALUE | 90% |
| POSITIVE PREDICTIVE VALUE | 90% |

Further, as shown in Table 3, specificity was 88% and predictive values were 90%. These results established the unexpected advantages of the ORAFUSION methods of this invention. Further, these results validated the designation of FDA breakthrough status for the methods claimed herein under the U.S. FD&C Act.

### Example 2: Validated Ligand-Binding Assays for Lateral Flow Reagents

Antigens and antibodies were characterized by the following methods: SDS-PAGE, LC-ESMS, ELISA, and SPR (Biacore). The SDS-PAGE and LC-MS were used to characterize the molecular weight and purity of the reagents. The ELISA and SPR were used to characterize the binding of the antigen-antibody complex. The LC and ESMS techniques showed the purity of the critical reagents were sufficient to develop the lateral flow assays. SDS-PAGE showed that these antibodies and reagents for lateral flow development were sufficiently pure for developing the lateral flow immunoassay. ELISA sandwich assays were used as a screening tool to select the best antibody pair for lateral flow immunoassay. The ELISA test showed that two antibodies could pair to the same antigen. The SPR demonstrated that the antibodies bound to different epitopes on the antigens and the antibodies were a good binding pair for the antigens.

### High Performance Liquid Chromatography and Electro-Spray Mass Spectroscopy

High Performance Liquid Chromatography (HPLC) and Electro-Spray Mass Spectroscopy (ESMS) were used for p16INK4a and sEGFR.

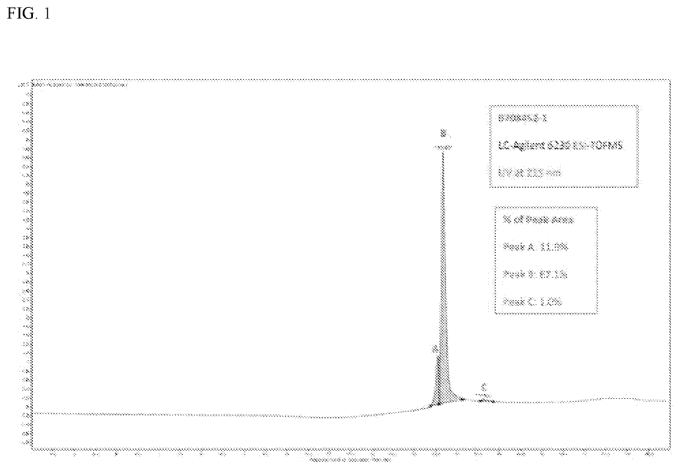

The p16INK4a and sEGFR were analyzed by LC-ESMS. As shown in FIG. 1, analysis indicated that the HPLC of p16INK4a/CDK2a (Peprotech) had three peaks. The first peak was 11.9% with a molecular weight (MW) of 16,548 g/mol, the second peak was 87.1% with a MW of 16,532 g/mol, and the third peak was 1.0%, a quantity too small to measure by ESMS. The difference between the native p16INKA/CDKN2a and the second peak was 16 g/mol. Thus, the LC of p16INK4a exhibited two peaks of 87.1% and 11.9%, which differed only by a molecular weight of 16 Da. Such small molecular weight change (0.1%) was not expected to affect the overall binding strength of the two antibodies to different epitopes on the antigen. The purity of this commercial antigen was useful for ELISA assays, SPR measurements, and LFIA strip development.

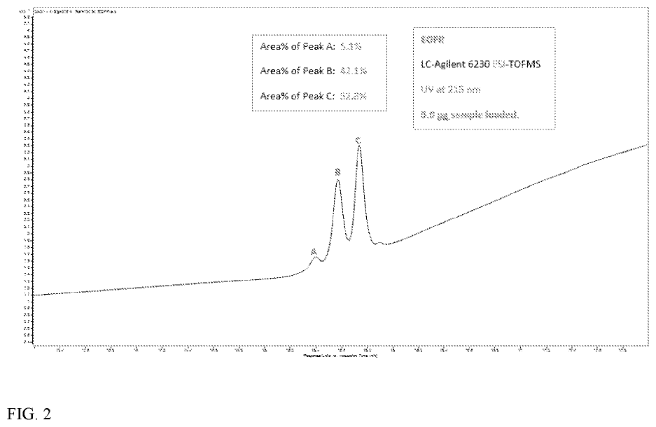

FIG. 2 shows the ES-MS of p16INK4a/CKD2a (PeproTech).

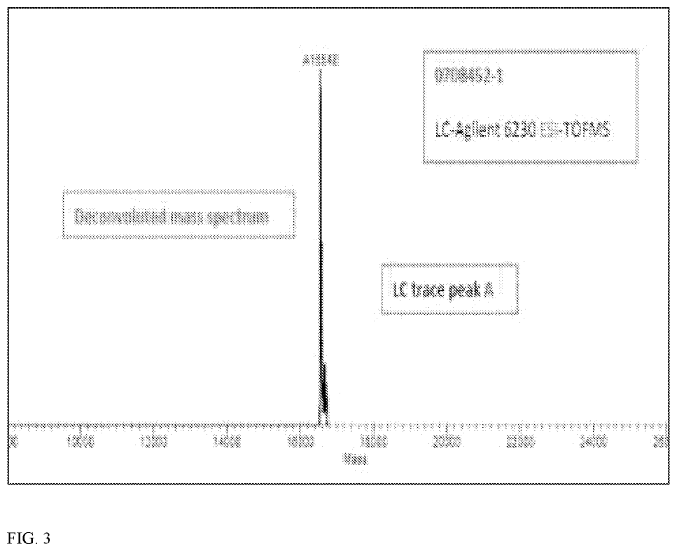

FIG. 3 shows that the HPLC chromatograph of sEGFR (R&D Systems) exhibited three peaks. The primary peak (C) was 82.5%, the second peak (B) was 12.4%, followed by the third peak (A) of 5.1%.

The sEGFR protein was too large to measure by ES-MS. Enzyme digestion on the sEGFR protein was used to identify a representative portion of fragments which aligned to the sequence of EGFR.

### SDS-PAGE Analysis of Antigens and Antibodies

As used herein, the extracellular domain of the epidermal growth factor receptor is abbreviated as sEGFR. The cetuximab antibody is referred to as sEGFRmAb-1. The EGFR. 1 antibody is referred to as sEGFRmAb-2. The antibodies for p16INK2a/CDK2a are referred to as p16mAb-1 and p16 mAb-2.

Pre-cast gels were used and inserted into the Mini-PROTEAN Tetra Cell according to the instructions of the manufacturer. All the running buffers and sample buffers were made according to the manufacturer's instructions. The proteins were run on two different types of gels: 1. A denatured gel; and 2. A reduced gel.

The SDS-PAGE analysis of p16INK4a detector and capture antibodies, and rhEGFR detector and capture antibodies used added proteins as follows: 1. Empty; 2. P16mAb961 (lane 2); 3. P16mAb695 (lane 3); 4. Empty; 5. rhEGFR antigen (lane 5); 6. EGFRAb1 (lane 6); and 7. EGFRAb2 (lane 7). A Peacock molecular weight ladder was used.

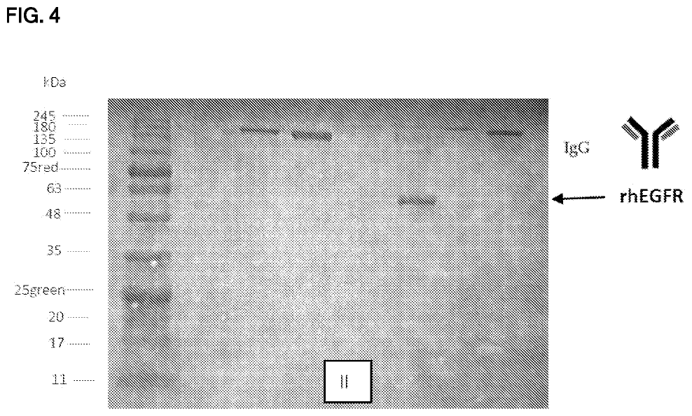

FIG. 4 shows the results of the gel II. The proteins run on the denatured gel (II) travelled through the gels intact. Therefore, the molecular weights of the bands indicate the intact molecular weight of the protein.

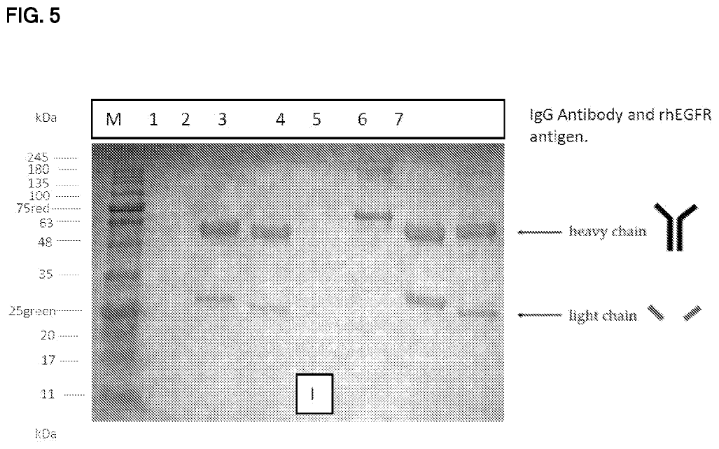

FIG. 5 shows the results of the gel I. The proteins run on the reduced gel travelled through the gels (I) as fragments of the full proteins. The fragments were parts of the protein that were connected by disulfide bonds to make the full proteins.

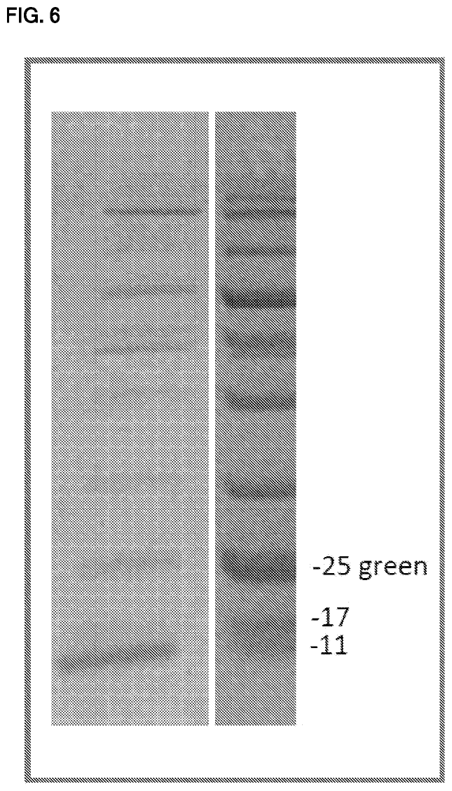

FIG. 6 shows SDS-PAGE gel of p16INK4A. The p16INK4A was pure as shown by comparison to the faint lines on the left which are Peacock molecular weight standard markers. The Peacock molecular weight standard marker was placed adjacent to the p16INK4a column for comparison (leak over from the ladder well). Only the molecular weights of the bottom three markers were added to the picture, but the ladder molecular weights were identical to the ladder in FIGS. 4 and 5.

### Enzyme-Linked Immunosorbent Assays

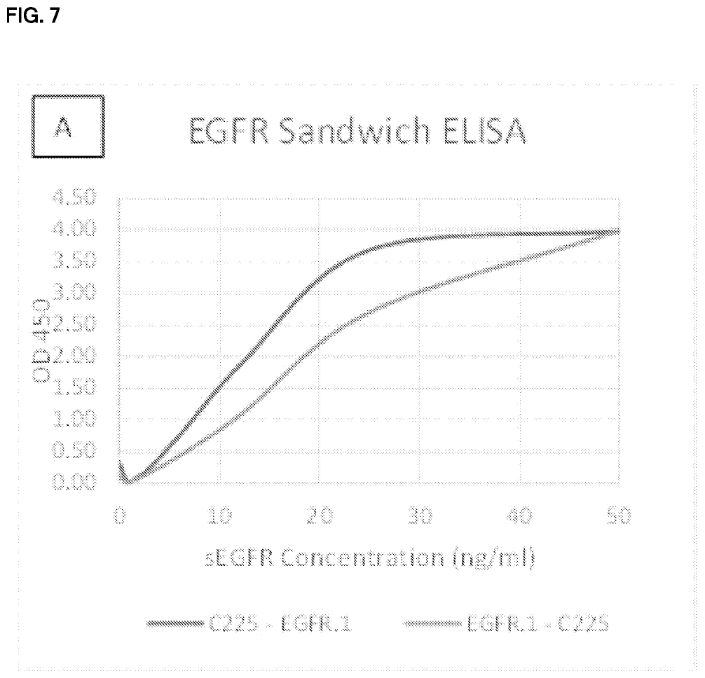

Sandwich ELISA were performed to determine the binding of sEGFR protein and the antibody pairs, C225/cetuximab (or EGFRAb1) and EGFR. 1 (or EGFRAb2). In the first ELISA test, C225 was coated to the bottom surface of the well, then sEGFR protein was added, followed by the addition of the monoclonal antibody EGFR. 1. The binding curve for this triplex of molecules is shown in FIG. 7 (upper curve).

In the second ELISA test, the monoclonal antibody pairs were added to the well in reverse order. The EGFR. 1 was first coated on the bottom of the well of the microtiter plate followed by the addition of the sEGFR protein. Once the sEGFR protein had bound to EGFR. 1 monoclonal antibody, then the second antibody, the C225, was added to the well. The results for this binding curve is shown in FIG. 7 (lower curve).

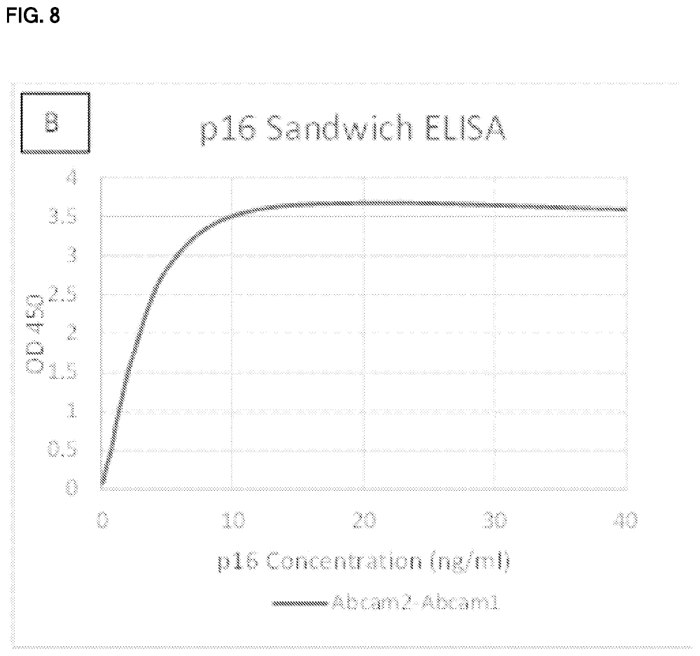

A sandwich ELISA was performed for p16 protein, while Abcam-2 antibody (Capture—ab244695) was coated on the ELISA plates and Abcam-1 (Detector—Ab244961) was used as the detector antibody. The results for this binding curve are shown in FIG. 8.

Surface Plasmon Resonance (SPR) sEGFR Sandwich Assay

In this study, the binding of two anti-sEGFR mAbs pairs to a single molecule of sEGFR was demonstrated by developing a sandwich type assay using a Biacore SPR instrument. The sandwich assay developed demonstrated that the mAbs were binding to different epitopes on the antigen surface.

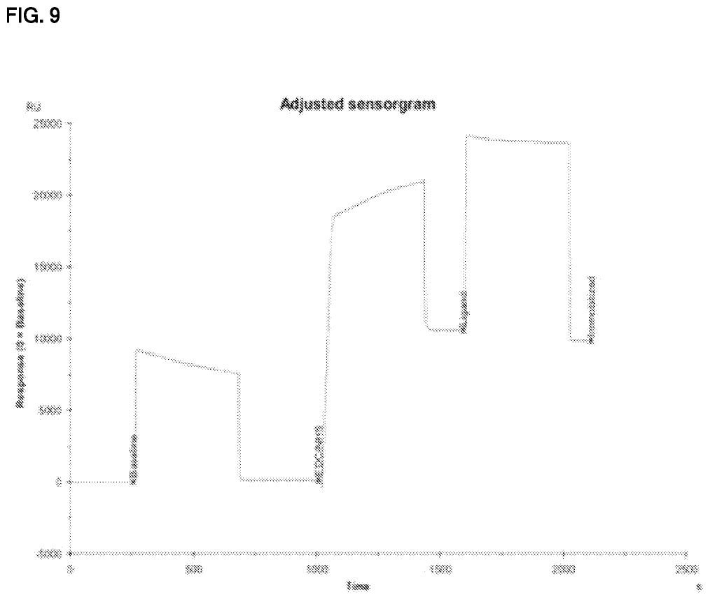

FIG. 9 shows a sensorgram of the immobilization of the anti-EGFR mAb (Cetuximab) to the surface of the CM5 chip.

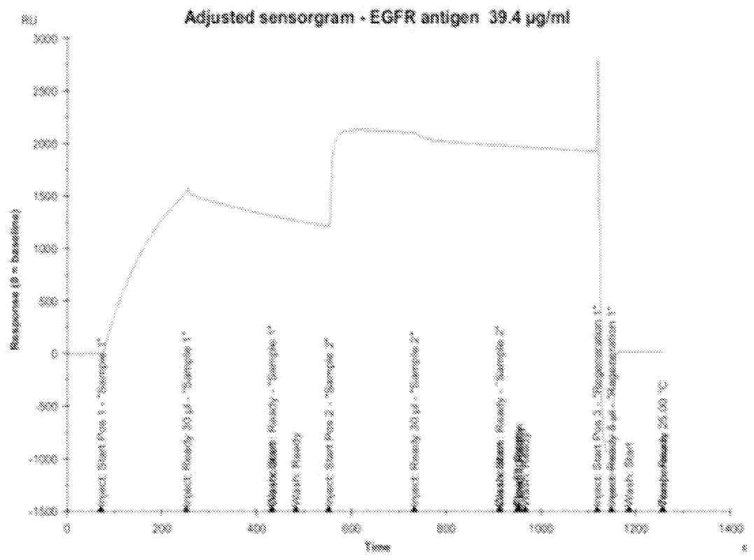

FIG. 10 shows a sensorgram of the sequential binding of rhEGFR to the CM5 chip bound anti-EGFR mAb (Cetuximab), followed by the binding of the anti-EGFR mAb (EGFR.1) to a second epitope on rhEGFR, forming the triplex of two antibodies bound to separate epitopes on the antigen, a standard sandwich assay configuration. This quantitatively shows the associative binding between the antibody and the antigen, the raising line, to the equilibrium state, the flat line, and finally, the binding of the secondary antibody, the second raising line, to the equilibrium state. After the equilibrium the dissociation of the antigen and secondary antibody from the primary antibody occurs in the regeneration step.

p16INK4a/mAbs SPR Sandwich Assay

In this study, the measure of a pair of anti-p16INK4a/CDK2a mAbs to a single molecule of p16INK4a was demonstrated by developing a sandwich type assay on the surface of a CM5 chip.

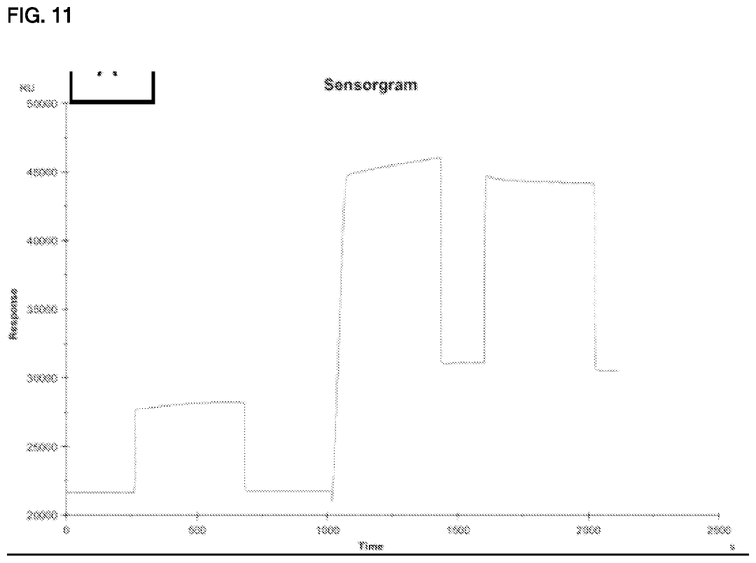

FIG. 11 shows a sensorgram of the immobilization of p16mAb695 to the activated surface of the CM5 chip. This sensorgram shows the activation, the attraction, and the covalent coupling of the p16mAb695 to the chip surface, and then the deactivation of the surface. The final height differential between the starting resonance units (RU) and the final RU indicates the degree of immobilization. The binding capacity of the surface depends on the concentration of immobilized ligand.

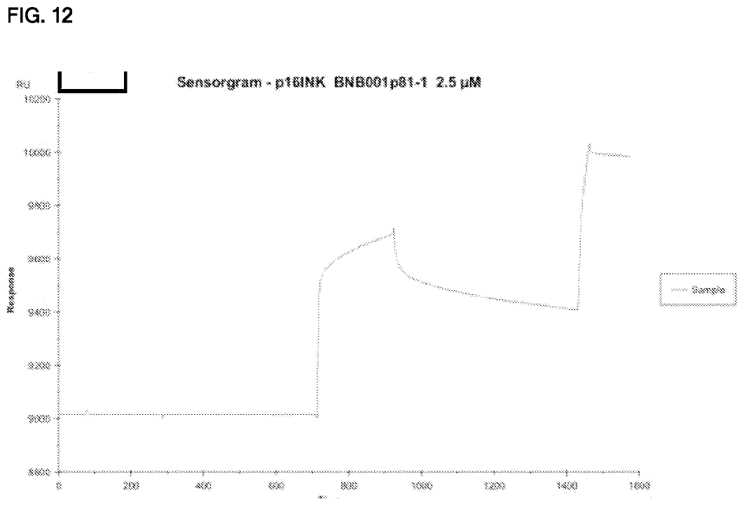

FIG. 12 shows a sensorgram of the sequential binding of p16INK4a to the immobilized, CM5 chip-bound antibody p16mAb695, followed by the sequential binding of the p16mAb961 antibody to a second epitope on the bound p16INK4a antigen, forming a triplex of a sandwich assay.

The sensorgrams of FIGS. 11 and 12 demonstrate that the pair of antibodies are binding to different molecular surfaces on the antigen. The different surfaces are typically classified as epitopes.

### Example 3: Materials and Reagents Used for Lateral Flow Biomarker Strips

Components for an embodiment of a lateral flow immunoassay device are shown in Table 4.

TABLE 4
Materials and reagents used for lateral flow biomarker strips

| Component | P16 | EGFR | pH |
|---|---|---|---|
| Sample | different matrices -Whole | different matrices - Whole | 7.0 |
| | human saliva and synthetic oral | human saliva and synthetic oral | |
| | fluid | fluid | |
| Protein | rhp 16 (Peprotech) | rhEGFR (R&D Systems, cat: | |
| | | 1095-ER-002, 10 ug/mL) | |
| Cassette | dual strip cassette | dual strip cassette | |
| Det-Ab | p16 mAb/IgG | Cetuximab (C225, Leinco | |
| | (Ab244961) (P16-1- Abcam, | Technologies) | |
| | Ab244695, rec. anti- | | |
| | CDKN2A/p16 antibody- | | |
| | capture) | | |
| Cap-Ab | p16 mAb/IgG | EGFR.1 (Lt600, In Vivo Gold | |
| | (Ab244695) (P16-2 - Abcam, | Functional Grade, Leinco) | |
| | Ab244961, rec. anti- | 1 mg/mL in PBS | |
| | CDKN2A/p16 antibody- | | |
| | detector) | | |
| Label | Gold-NS conjugate; dia = 150 | Gold-NS conjugate; | |
| | nm | 150-nm | |
| | mAb Loading = 20 ug/mL | mAb Loading = 20 ug/mL | |
| | [GNS] = 20 O.D. | [GNS] = 20 O.D. | |
| Sample | GFDX (untreated) | GFDX; treated (600 mM Tris, | |
| Pad/Buffer | | 3x PBS, 3% PVP-40, 1% BSA, | |
| | | 1% Casein, 2% Tween20, | |
| | | 0.05% Sodium Azide) | |
| Conj. Pad | GFDX: treated with mAb-GNS | GFDX: treated with mAb-GNS | |
| Sample vol | Sample - 50 uL | Sample - 50 uL | 9.0 |
| (uL) | Sample buffer vol. = 50 uL | Sample buffer vol. = 50 uL | |
| Membrane | MDI-150 | MDI-150 | |
| | (Adv. Microdevices PVT) | (Adv. Microdevices PVT) | |
| | 1 mg/mL | 1 mg/mL | |
| Test line | P16 mAb | EGFR.1 | |
| | (Ab244961) | (E336, Leinco) | |
| | Dispense rate = 0.08 ul/mm | Dispense rate = 0.08 ul/mm | |
| | [mAb] = 1 mg/mL; | [mAb] = 1 mg/mL; | |
| | 9-mm from bottom | 9-mm from bottom | |
| Control line | Donkey anti-Rabbit antibody | Goat anti-Mouse | |
| | (Lampire) | Dispense rate = 0.08 ul/mm | |
| | Dispense rate = 0.08 ul/mm | [mAb] = 1 mg/mL. | |
| | [mAb] = 1 mg/mL. | 15-mm from bottom | |
| | 15-mm from bottom | | |
| Absorbent/ | Ahlstrom | Ahlstrom | 9.0 |
| wicking pad | (thickness = 0.83 mm) | (thickness = 0.83 mm) | |
| Backing card | PDC, LLC, (custom made) | PDC, LLC (custom made) | |
| Running | 50 mM Borate, 1% PEG, 15% | 500 mM Tris, 0.2% 10 G, | 8.5 |
| buffer | Sodium deoxycholate | 0.35% Tween20, 0.25% PVP40, | |
| | | 8% SDC | |
| Testing | Dry Down | Dry Down | |
| format | | | |
| Run time | 20 | 20 | |
| (min) | | | |

### Example 4: Accurate P16 and EGFR Biomarker Concentrations were Measured with Synthetic Saliva Samples

The results of this experimental study showed that the lateral flow strips for p16 and EGFR were surprisingly reliable used with synthetic oral fluid.

This experiment was done using synthetic oral fluid (UTAK) as the matrix to determine the limit of detection (LoD) for the biomarkers, EGFR and p16. In the procedure, the biomarkers, p16 and EGFR, were added to the strip 2× the concentration of their LoD in synthetic oral fluid.

Curves were generated by measuring different concentrations of p16 and EGFR added to lateral flow immunoassay strips. The strips were read on a LUMOS LEELU reader.

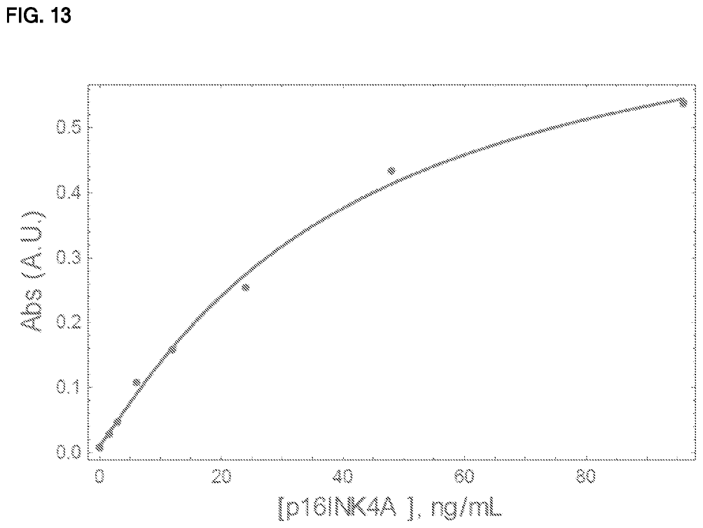

FIG. 13 shows the dose-response curve of p16 spiked in synthetic saliva. The dose-response curve of p16 used spiked pooled whole human saliva which was filtered (POREX SALETTO Oral Fluid Collection Device) (n=3). The biomarker, p16, was spiked into synthetic oral fluid (UTAK) and had a measuring interval from 0-96 ng/ml. The LoD for p16 was determined to be 1.5 ng/ml. In FIG. 13, the filled circles are data points measured by the reader, and the curve fitting line was calculated by a four (4) parameter logistics equation.

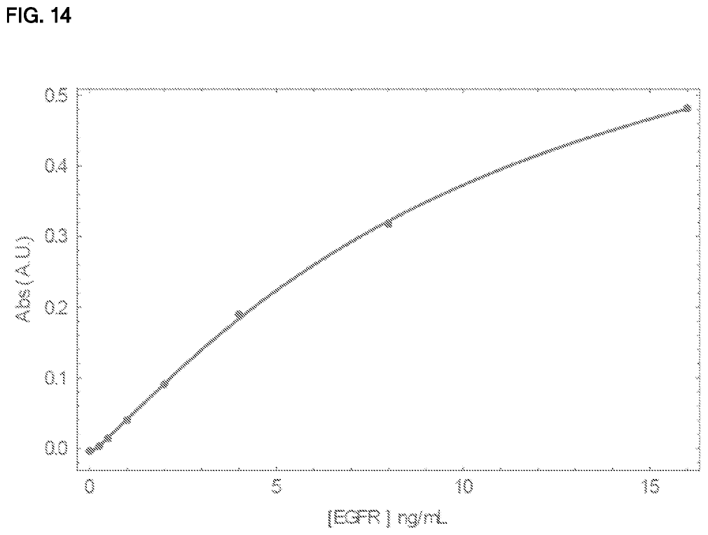

FIG. 14 shows the dose-response curve for EGFR spiked in synthetic saliva. The dose-response curve of EGFR used spiked pooled whole human saliva which was filtered (POREX SALETTO Oral Fluid Collection Device) (n=3). The antigen, EGFR, was spiked into synthetic oral fluid (UTAK) and had a measuring interval from 0-16 ng/ml. The LoD for EGFR was determined to be 1.0 ng/ml. In FIG. 14, the filled circles are data points measured by the reader, and the curve fitting line was calculated by a four (4) parameter (a, b, c, d) logistics equation. The % CV (not shown) varied from 10-25%.

The dose response parameters for biomarkers p16 and EGFR in synthetic oral fluid obtained by curve-fitting are shown in Table 5.

TABLE 5
Dose response parameters for biomarkers p16 and EGFR

| | a | d | c | | |
|---|---|---|---|---|---|
| Antigen | (ng/mL) | (ng/mL) | (ng/mL) | b | R Square |
| P16 | 0.01 ± 0.01 | 0.76 ± 0.13 | 41.33 ± 13.96 | 1.11 ± 0.20 | 0.9959 |
| EGFR | 0.0 | 0.56 ± 0.23 | 6.56 ± 0.43 | 1.35 ± 0.04 | 0.9999 |

The regression curve was a surprisingly close fit to the data as shown by R Squared values of 0.9959 and 0.9999 for p16 and EGFR, respectively.

The 5th, 50th, and 95th percentiles were calculated for each biomarker and are shown in Table 6. At the concentrations examined, a hook effect was not observed. The lateral low strips for EGFR and p16 were in the biologically relevant range. The p16 range in the C5th to C95th percentile intervals was from 18.37 to 64.29 ng/ml, which is in the biologically relevant range (0-160 ng/mL based on ELISA testing). The EGFR biomarker concentration for the C5th to C95th percentile intervals was from 5.85 to 7.27 ng/ml, which was in the biologically relevant range (0-25 ng/ml, based on ELISA testing) for EGFR in whole human saliva.

TABLE 6
Dose-response results for p16 and EGFR in pooled saliva

| | LoB | LoD | C5 | C50 | C95 | Hook |
|---|---|---|---|---|---|---|
| Antigen | (ng/mL) | (ng/mL) | (ng/mL) | (ng/mL) | (ng/mL) | (ng/mL) |
| P16 | 0.008 | 1.5 | 18.37 | 41.33 ± 13.95 | 64.29 | n.o. |
| | | (n ± 10) | | | | |
| EGFR | 0.014 | 1.0 | 5.85 | 6.56 ± 0.43 | 7.27 | n.o. |
| | | (n ± 10) | | | | |

In another study, the performance characteristics of the biomarker measurements based on artificial saliva (Synthetic Oral Fluid, UTAK) are shown in Table 7.

TABLE 7
Biomarker analytical performance characteristics
with artificial saliva

| Analytical Performance | | | | |
|---|---|---|---|---|
| Characteristic | P16 | | EGFR | |
| Analytical Sensitivity | 1.5 | ng/ml | 1 | ng/ml |
| Analytical Specificity | >95% | | >95% | |
| Trueness (Bias) | ±20% (est) | | ±20% (est) | |
| Precision (Reproducibility) | ±20% (est) | | ±20% (est) | |
| Accuracy | 80% | | 80% | |
| Limit of Detection | 1.5 | ng/ml | 1 | ng/ml |
| Sample | saliva | | saliva | |
| Measuring Range | 1.5 to 96 | ng/ml | 1.0 to 16 | ng/ml |
| Cut-off | 15 | ng/ml | 0.9 | ng/ml |

| | | |
|---|---|---|
| Linearity | R² = 0.9959 | R² = 0.9997 |
| Protocol | ELISA | ELISA |

### Example 5: Interference and Cross-Reactivity for Measurement of Biomarkers EGFR and P16 was Advantageously Eliminated

This example shows that interference and cross-reactivity of various endogenous and exogenous substances with measurement of biomarkers EGFR and P16 in a lateral flow assay was identified and eliminated.

Interference and cross reactivity studies were performed to determine the biomarker analytical specificity in the presence of other substances/agents in saliva.

Interference testing was used to observe any diminished test line intensity in the presence of the interfering substances, which were first mixed with biomarkers p16 and EGFR and then added to the oral synthetic fluid (UTAK).

Cross-reactivity testing was used to observe any decrease in the appearance of a test line when p16 or EGFR was spiked into the synthetic oral fluid (UTAK). The interfering substances were mixed in the synthetic oral fluid.

The interferents and cross-reacting substances or agents were any of (a) endogenous substances present in a patient's mouth, such as proteins and cellular elements, (b) exogenous substances ingested or used by a patient, such as alcohol, toothpaste, and mouthwash, and (c) pathogens that may infect the tissues in the oral cavity of the patient.

The lateral immunoassay strips were arranged according to NCX protocols. The cross-reactivity substances were diluted in synthetic oral fluid. The interferent substances were diluted in synthetic oral fluid along with the antigens P16 or EGFR. Then, the spiked samples were filtered through the POREX SALETTO Oral Fluid Collection Device. An aliquot was mixed with the lateral flow running buffer and applied to the strip and the test line signal was compared to the control test line. The control test line contained only the biomarker without cross-reactivity and interfering substances.

The proteins and cellular element substances tested were: bilirubin, 0.15 mg/mL, a-amylase, 250 ug/mL, human serum albumin (HAS), 15 mg/mL, IgA, 1 mg/mL, lactoferrin, 0.1 mg/mL, and whole blood, 4% v/v. No significant cross-reactivity was observed for any of these substances, except HSA for EGFR.

The ingested substances tested were: alcohol, coffee, mouthwash, and toothpaste, all at 6% v/v. No significant interference or cross-reactivity was observed for any of these substances, except coffee interfered with EGFR, mouthwash cross-reacted with P16, and toothpaste interfered with EGFR.

The pathogenic substances tested were: pooled bacteria, 10\hat{ }6 cfu, Candida albicans, 10\hat{ }6 cfu, and pooled viruses, 10\hat{ }5 cfu. No significant interference or cross-reactivity was observed for any of these substances, except Candida albicans interfered with P16. Pooled bacteria were Pseudomonas aeruginosa, Streptococcus mutans, and Eikenella corrodens Z22 (Zeptometrix, Buffalo). Pooled virus was Cytomegalovirus (CMV) (AD-169), Herpes Simplex Virus (Type-1), and Epstein-Barr Virus (B95-8).

For this study, a change in the intensity of a test line compared to the control test line of less than 30% was considered insignificant in cross-reactivities and interferences. Also, if the intensity of the sample test line and the control test line were less than 0.03 A.U. (LoD), the substances were considered insignificant in cross-reactivities and interferences.

According to this study, patients should fast for about one hour before taking the test. Patients should not have bleeding in the oral cavity. Patients should not have Candida Albicans in the oral cavity upon visual inspection.

### Example 6: Accurate P16 and EGFR Biomarker Concentrations were Measured with Pooled Freeze-Thaw Whole Human Saliva Samples

This example shows that pooling of whole human saliva samples can be used to improve precision of the biomarker measurements.

Thawed whole human saliva was collected, pooled and spiked with biomarkers EGFR and p16 to create dose-response curves. Pooling saliva reduces the variability for individual samples of saliva. Pooling of saliva was used for developing sample buffers and running buffers for the EGFR and p16 lateral flow strips.

Thirty (30) negative whole human saliva samples were pooled and spiked with varying concentrations of antigens. The thirty (30) negative defrosted whole human saliva samples were pooled and then spiked with varying ranges of concentrations of the antigens, p16 and EGFR. The samples were then filtered and diluted 1:1 in sample buffer. After being diluted 100-mL was aliquoted into the device of this invention and the sample was developed for 20 min before being read on a Lumos Leelu reader. The results of the absorbance (A.U.) were plotted against the spiked concentrations of the antigens.

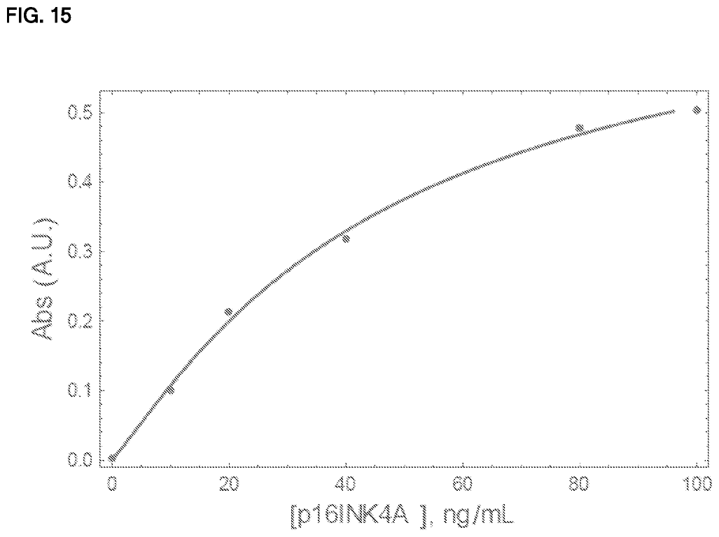

FIG. 15 shows the dose-response curve of p16 spiked in pooled whole human saliva and filtered (POREX SALETTO Oral Fluid Collection Device) (n=3).

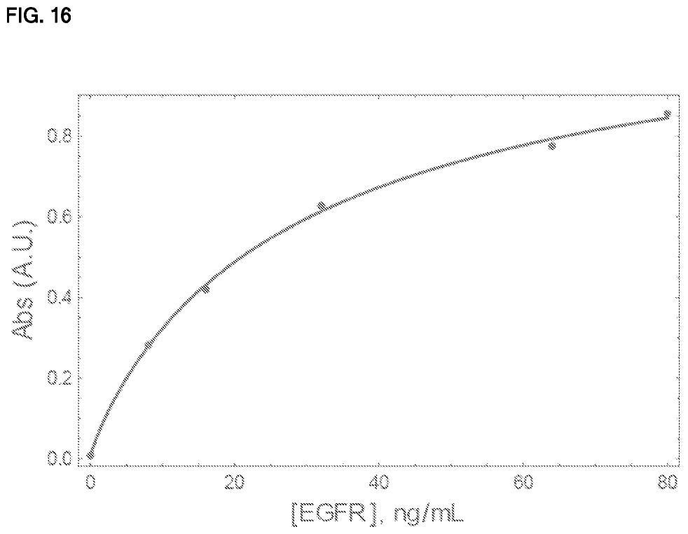

FIG. 16 shows the dose-response curve of EGFR spiked in pooled whole human saliva and filtered (POREX SALETTO Oral Fluid Collection Device) (n=3).

Parameters for logistic regression of the non-linear dose-response curve for p16 and EGFR in pooled saliva are given in Table 8. The regression curve was a surprisingly close fit to the data as shown by R Squared values of 0.9977 and 0.9988 for p16 and EGFR, respectively.

The dose response parameters for biomarkers p16 and EGFR spiked in pooled whole human saliva obtained by curve-fitting are shown in Table 8.

TABLE 8
Dose response for biomarkers p16 and EGFR spiked in pooled human saliva

| | a | d | c | | |
|---|---|---|---|---|---|
| Antigen | (ng/mL) | (ng/mL) | (ng/mL) | b | R Square |
| P16 | 0.001 ± 0.02 | 0.72 ± 0.14 | 46.84 ± 18.27 | 1.14 ± 0.23 | 0.9977 |
| EGFR | 0.007 ± 0.02 | 1.20 ± 0.20 | 30.74 ± 13.112 | 0.90 ± 0.17 | 0.9988 |

The regression curves were surprisingly close fit to the data as shown by R Squared values of 0.9977 and 0.9988 for p16 and EGFR, respectively. This shows that the biomarker measurements in frozen, thawed oral fluid were surprisingly reliable, so that unexpectedly high precision was obtained. Because of the high precision, even the potential effects of cross-reactivity or interference in a lateral flow immunoassay were determined. Further, the shapes of the curves showed there is an accurate relationship between the concentrations of the biomarkers and their absorbance values.

The 5th, 50th, and 95th percentiles were calculated for each biomarker and are shown in Table 9. The lateral low strips for EGFR and p16 were in the biologically relevant range. The p16 range in the C5th to C95th percentile intervals was from 16.82 to 76.86 ng/ml, which is in the biologically relevant range (0-160 ng/ml based on ELISA testing). The EGFR biomarker concentration for the C5th to C95th percentile intervals was from 9.167 to 52.31 ng/mL, which was in the biologically relevant range (0-25 ng/ml, based on ELISA testing) for EGFR in whole human saliva.

TABLE 9
Dose-response results for p16 and EGFR in thawed pooled human saliva

| | LoB | LoD | C5 | C50 | C95 | Hook |
|---|---|---|---|---|---|---|
| Antigen | (ng/mL) | (ng/mL) | (ng/mL) | (ng/mL) | (ng/mL) | (ng/mL) |
| P16 | n.d. | n.d. | 16.82 | 46.84 ± 18.25 | 76.86 | >100 |
| EGFR | n.d. | n.d. | 9.167 | 30.74 ± 13.11 | 52.31 | >80 |

### Example 7: Accurate P16 and EGFR Biomarker Concentrations were Measured with Pooled Fresh Saliva Samples

Fresh whole human saliva samples were pooled and then spiked with varying concentrations of the biomarkers, p16 and EGFR. The samples were then filtered and diluted 1:1 in sample buffer. After being diluted, 100 μL was aliquoted into a dual cassette well and the sample was developed for 20 min before being read on a Lumos Leelu reader. The results of the absorbance (A.U.) were plotted against the spiked concentrations of the antigens.

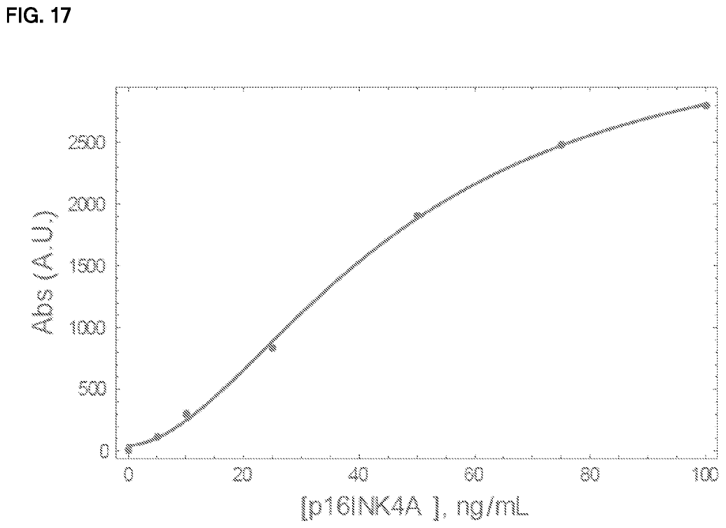

FIG. 17 shows the dose-response curve of p16 spiked in pooled whole human saliva, which was filtered (POREX SALETTO Oral Fluid Collection Device) (n=2).

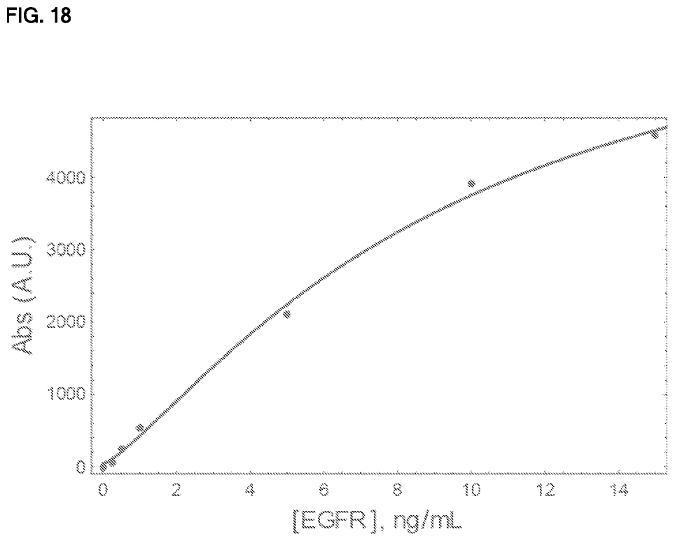

FIG. 18 shows the dose-response curve of EGFR spiked in pooled whole human saliva, which was filtered (POREX SALETTO Oral Fluid Collection Device) (n=2).

The curve fitted binding curve for the data in FIG. 17 is shown in Table 10.

TABLE 10
Dose response for p16 and EGFR spiked
in fresh pooled human saliva

| | a | d | c | | R |
|---|---|---|---|---|---|
| Antigen | (ng/mL) | (ng/mL) | (ng/mL) | b | Square |
| P16 | 45.1 ± 43.1 | 3500 ± 271 | 46.7 ± 4.78 | 1.81 ± | 0.9989 |
| | | | | 0.20 | |
| EGFR | 39.8 ± 117 | 7210 ± 1960 | 9.46 ± 4.25 | 1.28 ± | 0.9988 |
| | | | | 0.32 | |

The regression curve was a surprisingly close fit to the data as shown by R Squared values of 0.9989 and 0.9988 for p16 and EGFR, respectively. This shows that the biomarker measurements in fresh oral fluid were surprisingly reliable, so that unexpectedly high precision was obtained. Because of the high precision, even the potential effects of cross-reactivity or interference in a lateral flow immunoassay were determined. Further, the shapes of the curves showed there is an accurate relationship between the concentrations of the biomarkers and their absorbance values.

The 5th, 50th, and 95th percentiles were calculated for each biomarker and are shown in Table 11. The lateral low strips for EGFR and p16 were in the biologically relevant range. The p16 range in the C5th to C95th percentile intervals was from 38.81 to 54.53 ng/mL, which is in the biologically relevant range (0-160 ng/ml based on ELISA testing). The EGFR biomarker concentration for the C5th to C95th percentile intervals was from 2.26 to 16.49 ng/ml, which was in the biologically relevant range (0-25 ng/mL, based on ELISA testing) for EGFR in whole human saliva.

TABLE 11
Dose-response results for p16 and EGFR in fresh pooled human saliva

| | LoB | LoD | C5 | C50 | C95 | Hook |
|---|---|---|---|---|---|---|
| Antigen | (A.U.) | (ng/mL) | (ng/mL) | (ng/mL) | (ng/mL) | (ng/mL) |
| P16 | 45.09 ± 43.16 | 5.53 | 38.81 | 46.67 ± 4.78 | 54.53 | n.o. |
| EGFR | 39.85 ± 115.9 | 0.57 | 2.26 | 9.46 ± 4.253 | 16.49 | n.o. |

The fitting constant c was used to determine the C95, C50, and C5 percentiles. The fitting constant a was used to determine the limit of blank (LOB), and the limit of detection (LOD), according to Clinical Laboratory Standards Institute (CLSI) EP17-A12 instructions.

At the concentrations examined, a hook effect was not observed.

By comparison, the C50 values for p16 in frozen and fresh pooled whole human saliva were nearly identical, 46.8 and 46.7, respectively.

By comparison, the C50 values for EGFR in frozen and fresh pooled whole human saliva were significantly different, 30.7 and 9.5, respectively.

### Example 8: Clinical Performance Characteristics for Assessing Oral Cancer Risk Using Methods of this Invention were Highly Accurate

In another study, the likelihood ratios for testing based on the methods of this invention were surprisingly high, which showed that the assessment of oral cancer risk was highly accurate.

For this study, likelihood ratios were determined based on sensitivity/(1−specificity). The clinical performance characteristics of the saliva biomarker measurements are shown in Table 12. As shown in Table 12, the likelihood ratios were very high, which showed that the assessment of oral cancer risk was highly accurate.

TABLE 12
Clinical performance characteristics for biomarker measurements

| Clinical Performance Characteristic | P16 | | EGFR | |
|---|---|---|---|---|
| Threshold Value | 8-15 | ng/ml | 0.7-1.0 | ng/ml |
| Sensitivity | 88% | | 92% | |
| Specificity | 92% | | 100% | |
| Positive predictive value | 90% | | 100% | |
| Negative predictive value | 91% | | 94% | |
| Likelihood ratio | 12 | | >100 | |
| Expected Values in Normal | 7.7 | ng/ml | 0.2 | ng/ml |
| Populations | | | | |
| Expected Values in Affected | 60.9 | ng/ml | 3.6 | ng/ml |

Populations

### Example 9: High Sensitivity and Specificity were Achieved for Assessing Oral Cancer Risk Using a Lateral Flow Immunoassay Method of this Invention with Freeze-Thaw Whole Human Saliva

This example shows that the lateral flow immunoassay (LFIA) accurately provides positive and negative oral cancer risk readings using concentrations of the biomarkers P16 and EGFR. The stratification of oral cancer risk biomarker values was confirmed by comparison of measurements of whole human saliva samples with ELISA measurements and ANOVA analysis.

Biomarker concentration cut-off values for moderate oral cancer risk stratification and estimated clinical sensitivity and specificity results for biomarkers p16 and EGFR in whole human saliva were determined in a blinded study, which provided 50 positive and 67 negative results. Elevated risk, meaning a positive reading, would be indicated for values above the upper cut-off, and low risk, meaning a negative reading, would be indicated for values below the lower cut-off.

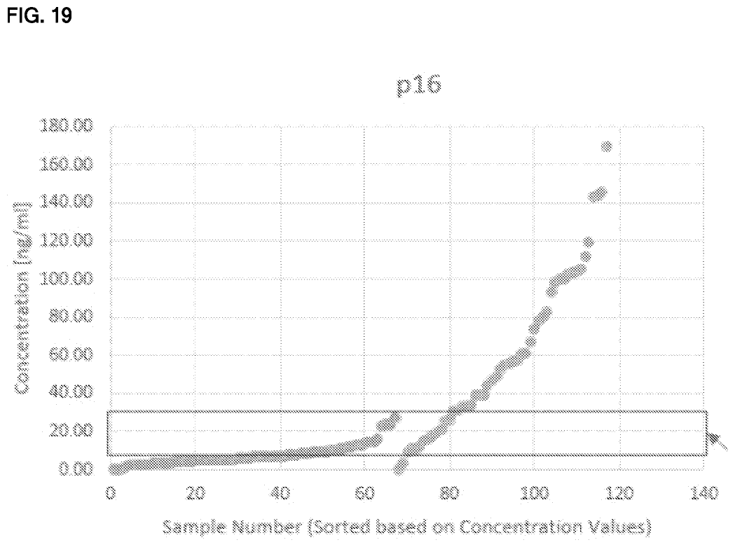

As shown in FIG. 19, for biomarker P16, the cut-off levels for moderate risk were 10.5 ng/ml and 27.5 ng/ml. The estimated sensitivity was 94% and the estimated specificity was 100%. The estimated specificity met the acceptance criteria of over 90%.

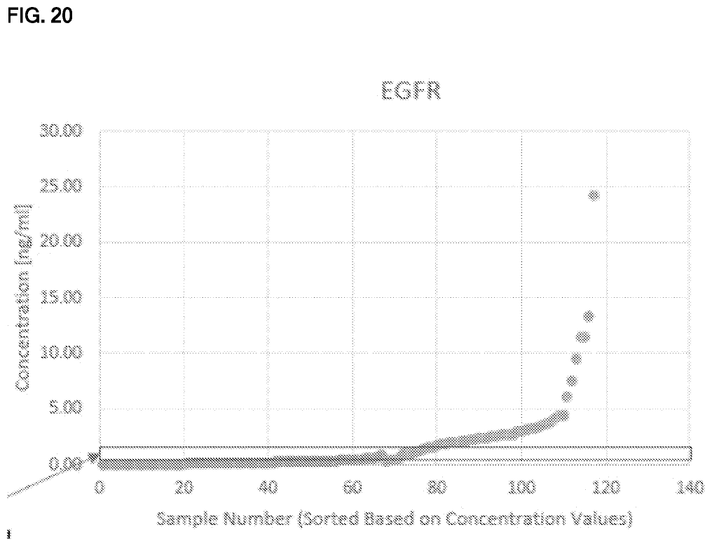

As shown in FIG. 20, for biomarker EGFR, the cut-off levels for moderate risk were 0.75 ng/ml and 1.4 ng/ml. The estimated sensitivity was 92% and the estimated specificity was 100%. The estimated specificity met the acceptance criteria of over 90%.

These results were unexpectedly advantageous for determining an oral cancer risk compared to any conventional parameters.

In FIGS. 19 and 20, the low region contains only negative results that match the pathology findings. The moderate region includes the range of individual ELISA results where positive and negative pathology confirmed results are overlapping. The elevated range contain only positive results that match the pathology findings.

### Example 10: Saliva Sample Freeze-Thaw and Refrigeration Stability

A saliva sample degradation and stability study was performed and demonstrated the effects of freeze-thaw and refrigeration cycle on the measured concentrations of biomarkers p16 and EGFR. Slight degradation was observed in samples stored more than 24 hours in the refrigerator or those that had undergone more than one cycle of freeze and thaw. Slight sample degradation was observed based on the concentration of p16, EGFR, a-amylase, and lysozyme.

For one cycle of freeze and thaw, P16 was found to retain 85% intensity (41.8/48.9, in ng/ml).

For one cycle of freeze and thaw, EGFR was found to retain 88% intensity (2.2/2.5, in ng/ml).

What is claimed is:

1. A method for assessing a risk level of, diagnosing, prognosing, or treating an oral cancer in a subject, the method comprising:
detecting biomarker levels in a saliva sample from the subject; and
assessing the risk level of the oral cancer using the biomarker levels in combination with clinical factors of the subject.

2. The method of claim 1, comprising treating the oral cancer based on the assessed risk level, diagnosis, or prognosis.

3. The method of claim 1, wherein the biomarkers comprise p16 and EGFR.

4. The method of any of claims claim 1, wherein the oral mucosal abnormality is an oral lesion, an oral cancer, a dysplasia, an early-stage cancer, or a pre-malignant disorder.

5. The method of claim 1, wherein the oral cancer is located in any of a lip tissue, a gum tissue, a jaw tissue, a tongue tissue, or an oropharyngeal tissue.

6. The method of claim 1, wherein the method is a point of care risk assessment test.

7. The method of claim 1, wherein the sample is obtained and the method is performed in a point of care setting.

8. The method of claim 1, wherein the method is performed in a point of care setting with a test duration of 3-5 minutes, or 5-50 minutes, or about 20 minutes.

9. The method of claim 1, wherein the saliva sample is prepared from filtered whole saliva in a buffer solution.

10. The method of claim 1, wherein the saliva sample is stored for less than 24 hours in a refrigerator, or is frozen and thawed no more than once.

11. The method of claim 1, wherein the method provides an early detection of oral cancer or a risk of oral cancer in a subject.

12. The method of claim 1, wherein the treating is any one or more of surgery, therapeutic radiation, chemotherapy, and drug therapy.

13. The method of claim 1, wherein the method reduces a need for invasive or debilitating cancer treatment.

14. The method of any one of claims 1-13, wherein the assessing a risk level of, diagnosing, prognosing, or treating comprises a logic combination of a sensor fusion function and a risk factor function to provide an output.

15. The method of any one of claims 1-13, wherein the method is performed after visualizing an oral mucosal abnormality in the subject by a trained healthcare provider.

16. The method of any one of claims 1-13, wherein the clinical factors comprise age, gender, alcohol use, tobacco use, and presence of a non-oral diagnosed cancer.

17. The method of any one of claims 1-13, wherein the wherein the assessing a risk level of, diagnosing, prognosing, or treating results in a greater sensitivity, specificity, or predictive value as compared to visual oral examination alone.

18. The method of any one of claims 1-13, wherein the assessing a risk level of, diagnosing, prognosing, or treating provides a specificity of at least 70%, or at least 80%, or at least 85%.

19. The method of any one of claims 1-13, wherein the assessing a risk level of, diagnosing, prognosing, or treating provides a sensitivity of at least 70%, or at least 80%, or at least 85%.

20. The method of any one of claims 1-13, wherein the assessing a risk level of, diagnosing, prognosing, or treating provides a negative or positive predictive value of at least 70%, or at least 80%, or at least 85%, or at least 90%.

21. The method of any one of claims 1-13, wherein p16 is detected in a biologically relevant range from 0 to 160 ng/mL, or in a range of C5th to C95th percentile from 18.37 to 64.20 ng/ml, respectively.

22. The method of any one of claims 1-13, wherein EGFR is detected in a biologically relevant range from 0 to 25 ng/mL, or in a range of C5th to C95th percentile from 2.43 to 16.50 ng/ml, respectively.

23. The method of any one of claims 1-13, wherein no significant interference is caused by bilirubin, a-amylase, IgA, lactoferrin, whole blood, pooled common bacteria, or pooled common viruses.

24. The method of any one of claims 1-13, wherein the level of p16 is detected as being low when less than 10.5 ng/mL, as being moderate when greater than 10.5 ng/ml and less than 27.5 ng/mL, and as being elevated when greater than 27.5 ng/ml.

25. The method of any one of claims 1-13, wherein the level of EGFR is detected as being low when less than 0.75 ng/ml, as being moderate when greater than 0.75 ng/ml and less than 1.4 ng/mL, and as being elevated when greater than 1.4 ng/ml.

26. The method of claim 14, wherein the sensor fusion function is based on two lateral flow biomarker levels being a low, moderate, or elevated level for p16 and a low, moderate, or elevated level for EGFR.

27. The method of claim 14, wherein the risk factor function is based on risk factors being subject age, gender, alcohol use, tobacco use, and presence of a non-oral diagnosed cancer, wherein the risk factor function is determined by logistic regression to provide three risk ranges being low, moderate, and elevated risk.

28. The method of claim 27, wherein the regression takes the highest value for a risk factor when the clinical data is not available.

29. The method of claim 14, wherein an output is a treatment referral based on the logic combination of the sensor fusion function and the risk factor function, wherein the output is one of:
no treatment referral due to low combined risk;
clinician decision for treatment referral due to moderate combined risk; or
treatment referral due to moderate or elevated combined risk.

30. The method of claim 29, wherein the subject is immediately referred for cancer treatment based on moderate or elevated combined risk.

31. A method for diagnosing, prognosing or monitoring an oral cancer in a subject, the method comprising:
measuring biomarker levels in a saliva sample from the subject;
comparing the levels of the biomarkers to reference levels based on a control group of subjects;
detecting differences in the biomarker levels between the subject and the control group;
diagnosing, prognosing or monitoring the oral cancer in the subject based on the differences.

32. The method of claim 31, wherein the biomarkers comprise p16 and EGFR.

33. The method of claim 31, wherein the oral mucosal abnormality is an oral lesion, an oral cancer, a dysplasia, an early-stage cancer, or a pre-malignant disorder.

34. The method of claim 31, wherein the oral cancer is located in any of a lip tissue, a gum tissue, a jaw tissue, a tongue tissue, or an oropharyngeal tissue.

35. The method of claim 31, wherein the method is performed in a point of care setting with a test duration of 3-5 minutes, or 5-50 minutes, or about 20 minutes.

36. The method of claim 31, wherein the saliva sample is prepared from filtered whole saliva in a buffer solution.

37. The method of claim 31, wherein the sample is stored for less than 24 hours in a refrigerator, or is frozen and thawed no more than once.

38. The method of claim 31, wherein the method provides an early detection of oral cancer or a risk of oral cancer in a subject.

39. The method of claim 31, wherein the method reduces a need for invasive or debilitating cancer treatment.

40. The method of claim 31, wherein the control group comprises subjects having an oral mucosal abnormality or oral cancer.

41. The method of claim 31, wherein the biomarker levels are determined by steps comprising:
detecting a test line and a control line on a lateral flow substrate;
determining an area for each of the test line and the control line and eliminating non-line areas; and
measuring the optical intensities of the test line and the control line.

42. The method of any one of claims 31-41, wherein the diagnosing or prognosing comprises a logic combination of a sensor fusion function and a risk factor function to provide an output.

43. The method of any one of claims 31-41, wherein the method is performed after visualizing an oral mucosal abnormality in the subject by a trained healthcare provider.

44. The method of any one of claims 31-41, wherein diagnosing or prognosing is based on additional clinical factors comprising age, gender, alcohol use, tobacco use, and presence of a non-oral diagnosed cancer.

45. The method of any one of claims 31-41, wherein the biomarker levels are determined by steps comprising:
detecting a test line and a control line on a lateral flow substrate;
determining an area for each of the test line and the control line and eliminating non-line areas; and
measuring the optical intensities of the test line and the control line.

46. The method of any one of claims 31-41, wherein the diagnosing or prognosing results in a greater sensitivity, specificity, or predictive value as compared to visual oral examination alone.

47. The method of any one of claims 31-41, wherein the diagnosing or prognosing provides a specificity of at least 70%, or at least 80%, or at least 85%.

48. The method of any one of claims 31-41, wherein the diagnosing or prognosing provides a sensitivity of at least 70%, or at least 80%, or at least 85%.

49. The method of any one of claims 31-41, wherein the diagnosing or prognosing provides a negative or positive predictive value of at least 70%, or at least 80%, or at least 85%, or at least 90%.

50. The method of any one of claims 31-41, wherein p16 is detected in a biologically relevant range from 0 to 160 ng/mL, or in a range of C5th to C95th percentile from 18.37 to 64.20 ng/mL, respectively.

51. The method of any one of claims 31-41, wherein EGFR is detected in a biologically relevant range from 0 to 25 ng/mL, or in a range of C5th to C95th percentile from 2.43 to 16.50 ng/ml, respectively.

52. The method of any one of claims 31-41, wherein no significant interference is caused by bilirubin, a-amylase, IgA, lactoferrin, whole blood, pooled common bacteria, or pooled common viruses.

53. The method of any one of claims 31-41, wherein the level of p16 is detected as being low when less than 10.5 ng/mL, as being moderate when greater than 10.5 ng/ml and less than 27.5 ng/mL, and as being elevated when greater than 27.5 ng/mL.

54. The method of any one of claims 31-41, wherein the level of EGFR is detected as being low when less than 0.75 ng/mL, as being moderate when greater than 0.75 ng/mL and less than 1.4 ng/mL, and as being elevated when greater than 1.4 ng/ml.

55. The method of claim 42, wherein the sensor fusion function is based on two lateral flow biomarker levels being a low, moderate, or elevated level for p16 and a low, moderate, or elevated level for EGFR.

56. The method of claim 42, wherein the risk factor function is based on risk factors being subject age, gender, alcohol use, tobacco use, and presence of a non-oral diagnosed cancer, wherein the risk factor function is determined by logistic regression to provide three risk ranges being low, moderate, and elevated risk.

57. The method of claim 56, wherein the regression takes the highest value for a risk factor when the clinical data is not available.

58. The method of claim 42, wherein an output is a treatment referral based on the logic combination of the sensor fusion function and the risk factor function, wherein the output is one of:
no treatment referral due to low combined risk;
clinician decision for treatment referral due to moderate combined risk; or
treatment referral due to moderate or elevated combined risk.

59. The method of claim 58, wherein the subject is immediately referred for cancer treatment based on moderate or elevated combined risk.

60. A method for data acquisition, the method comprising:
obtaining a saliva sample from a subject;
filtering the saliva sample;
adding the filtered saliva to a buffer solution; and
measuring biomarker levels in the filtered saliva sample.

61. The method of claim 60, wherein the biomarkers comprise proteomic p16 and EGFR.

62. The method of any one of claims 60-61, wherein the biomarker levels are measured on a lateral flow test strip using an optical reader.

63. The method of any one of claims 60-62, wherein the filtering removes particles having a range of size from 1 to 100 micrometers, or from 1 to 10 micrometers, or from 0.1 to 10 micrometers, or from 0.1 to 5 micrometers, or from 0.1 to 2 micrometers, or from 0.1 to 1 micrometers.

64. The method of any one of claims 60-62, wherein the filtering removes particles having a size greater than about 0.05 micrometers, or about 0.1 micrometers, or about 0.2 micrometers, or about 0.45 micrometers, or about 1 micrometers, or about 2 micrometers, or about 5 micrometers.

65. The method of any one of claims 60-64, wherein the saliva sample is stored for less than 24 hours in a refrigerator, or is frozen and thawed no more than once.

66. The method of any one of claims 60-65, wherein the buffer solution is filtered after adding the saliva.

67. The method of any one of claims 60-66, wherein the method is performed in a point of care setting with a duration of 3-5 minutes, or 5-50 minutes, or about 20 minutes.

68. The method of any one of claims 60-67, wherein biomarker p16 is detected in a biologically relevant range from 0 to 160 ng/mL, or in a range of C5th to C95th percentile from 18.37 to 64.20 ng/mL, respectively.

69. The method of any one of claims 60-67, wherein biomarker EGFR is detected in a biologically relevant range from 0 to 25 ng/mL, or in a range of C5th to C95th percentile from 2.43 to 16.50 ng/mL, respectively.

70. The method of any one of claims 60-67, wherein the measured biomarkers levels are accurate even in the presence of any one or more of bilirubin, a-amylase, IgA, lactoferrin, whole blood, pooled common bacteria, or pooled common viruses.

71. The method of any one of claims 60-67, wherein the level of biomarker p16 is detected as being low when less than 10.5 ng/mL, as being moderate when greater than 10.5 ng/mL and less than 27.5 ng/ml, and as being elevated when greater than 27.5 ng/ml.

72. The method of any one of claims 60-67, wherein the level of biomarker EGFR is detected as being low when less than 0.75 ng/mL, as being moderate when greater than 0.75 ng/ml and less than 1.4 ng/mL, and as being elevated when greater than 1.4 ng/mL.

73. The method of any one of claims 60-72, wherein the subject presents an oral mucosal abnormality viewed by a health care provider.

74. The method of claim 73, wherein the oral mucosal abnormality is an oral lesion, an oral cancer, a dysplasia, an early-stage cancer, or a pre-malignant disorder.

75. The method of claim 73, wherein the oral mucosal abnormality is located in any of a lip tissue, a gum tissue, a jaw tissue, a tongue tissue, or an oropharyngeal tissue.
